(19) United States
(12) Patent Application Publication
(10) Pub. No.: US 2005/0266464 A1
(43) Pub. Date: Dec. 1, 2005

(54) C1q family member proteins with altered immunogenicity

(75) Inventors: Shannon Alicia Marshall, San Francisco, CA (US); Gregory L. Moore, Pasadena, CA (US); Arthur J. Chirino, Camarillo, CA (US); John R. Desjarlais, Pasadena, CA (US)

Correspondence Address: Robin M. Silva;Dorsey & Whitney LLP, Intellectual Property Department, 555 California Street, Suite 1000, San Francisco, CA, 94104-1513, US

(73) Assignee: Xencor, Inc.

(21) Appl. No.: 11/132,162

(22) Filed: May 17, 2005

Related U.S. Application Data
(60) Provisional application No. 60/573,301, filed on May 21, 2004.

(51) Int. Cl. C12Q 1/68; A61K 38/17; C07K 14/74; C07H 21/04; C12P 21/06; C12N 15/09

(52) U.S. Cl. 435/6; 435/69.1; 435/320.1; 435/325; 530/350; 536/23.5; 514/12

(57) ABSTRACT

The present invention relates to non-naturally occurring variant C1q Super Family member proteins with reduced immunogenicity. More specifically, the present invention relates to variant adiponectin and CTRP1 proteins with reduced immunogenicity.

This application claims benefit under 35 U.S.C. §119(e) to U.S. Ser. No. 60/573,301, filed May 21, 2004, which is incorporated by reference in its entirety for all purposes.

### FIELD OF THE INVENTION

The present invention relates to variant C1q super family (“C1q SF”) member proteins with reduced immunogenicity. In particular, variants of adiponectin and CTRP1 with reduced ability to bind one or more human class II MHC molecules are described.

### BACKGROUND OF THE INVENTION

Immunogenicity is a major barrier to the development and utilization of protein therapeutics. Although immune responses are typically most severe for non-human proteins, even therapeutics based on human proteins may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population. Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. One special class of side effects results when neutralizing antibodies cross-react with an endogenous protein and block its function.

Adiponectin, also known as adipocyte complement-related protein of 30 kDa (ACRP30) is a secreted serum protein expressed exclusively in differentiated adipocytes. Strong sequence similarity exists between adiponectin and the three subunits of complement factor C1q, the Siberian chipmunk proteins HP-20, -25, and -27, CORS26, CTRP-5, and G-protein-coupled receptor interacting protein. All of the adiponectin homologs contain a similar modular structure comprising an N-terminal collagenous domain followed by a C-terminal globular trimerization domain. The crystal structure of the adiponectin globular trimer reveals an unexpected homology with the TNF family of cytokines. In spite of the lack of homology of the primary sequence, structural features between TNF-α and adiponectin are highly conserved. Adiponectin and TNF-α both trimerize via key conserved hydrophobic residues, both have a ten-strand jelly-roll folding topology, and both form bell-shaped homotrimeric oligomers. See, for example, Scherer et al., J. Biol. Chem. 270(45): 26746-9, 1995, entirely incorporated by reference.

Metabolic studies have demonstrated a role for adiponectin in the regulation of glucose and lipid homeostasis. Adiponectin increases insulin sensitivity by increasing tissue fat oxidation, resulting in reduced circulating fatty acid levels and reduced intracellular triglyceride contents in liver and muscle. This protein also suppresses the expression of adhesion molecules in vascular endothelial cells and cytokine production from macrophages, thus inhibiting the inflammatory processes that occur during the early phases of atherosclerosis.

Adiponectin has putative anti-hyperglycemic, anti-atherogenic, and anti-inflammatory properties and has potential utility in the treatment of diseases associated with insulin resistance, including type 2 diabetes mellitus, obesity, lipodystrophic disorders, and other conditions associated with the regulation of glucose or lipid metabolism. Adiponectin may also prove beneficial in the prevention of cardiovascular diseases, including atherosclerosis and coronary artery disease, and in the prevention and treatment of muscle disorders and liver diseases. See for example Berg et al. Trends Endocrinol. Metab. 13: 84-89 (2002), Diez and Iglesias Eur. J. Endocrinol. 148: 293-300 (2003), Xu et al. J. Clin. Invest. 112: 91-100 (2003), Patent WO-00192330, and Patent WO-02100427, all entirely incorporated by reference.

Two receptors for globular and full-length adiponectin have been identified, AdipoR1 and AdipoR2. AdipoR1 is abundantly expressed in skeletal muscle, whereas AdipoR2 is primarily expressed in the liver. These two receptors are predicted to contain seven transmembrane domains, but to be structurally and functionally distinct from G-protein-coupled receptors. Expression and suppression studies using small-interfering RNA suggest that these receptors mediate adiponectin's effects on fatty-acid oxidation and glucose uptake, as well as its increased AMP kinas.

C1q-TNF Related Protein 1 (CTRP1), also known as zsig37 and C1QTNF1, is highly expressed in endothelial and vascular smooth muscle cells and has been shown to bind to collagen exposed at sites of acute vascular injury. CTRP1 has been found to be a potent inhibitor of collagen-induced platelet activation and acts locally to prevent the formation of an artery-blocking clot at the site of vascular injury. CTRP1 treatment has been found to prevent the blockage of blood flow in animal models of plaque rupture, associated with heart attack and stroke, and models of vascular surgery, such as angioplasty. Unlike other agents used to inhibit thrombotic occlusion, CTRP1 has shown no significant systemic effect on blood coagulation when tested in animal models. Because of its potent effects in preventing platelet activation and arterial blockage and the apparent lack of bleeding complications induced by its administration, CTRP1 may have clinical utility in treating a variety of conditions associated with vascular damage including coronary angioplasty, carotid endarterectomy and stroke. See, for example, U.S. Pat. Nos. 6,803,450; 6,566,499; and 6,265,544, all entirely incorporated by reference. Other C1q SF members include CTRP2, CTRP3, CTRP4, CTRP5, CTRP6 and CTRP7. See Wong et al., PNAS, v. 110 no. 28: 10302-7 (2004), entirely incorporated by reference.

C1q SF members, like all proteins, have the potential to induce unwanted immune responses when used as a therapeutic. Accordingly, the development of therapeutics based on C1q SF members may be facilitated by pre-emptively reducing the potential immunogenicity of C1q SF members. Several methods have been developed to modulate the immunogenicity of proteins. In some cases, PEGylation has been observed to reduce the fraction of patients who raise neutralizing antibodies by sterically blocking access to antibody agretopes (see for example, Hershfield et. al. PNAS 1991 88:7185-7189 (1991); Bailon. et al. Bioconjug. Chem. 12: 195-202(2001); He et al. Life Sci. 65: 355-368 (1999), all entirely incorporated by reference). Methods that improve the solution properties of a protein therapeutic may also reduce immunogenicity, as aggregates have been observed to be more immunogenic than soluble proteins.

A more general approach to immunogenicity reduction involves mutagenesis targeted at the agretopes in the protein sequence and structure that are most responsible for stimulating the immune system. Some success has been achieved by randomly replacing solvent-exposed residues to lower binding affinity to panels of known neutralizing antibodies (see for example Laroche et. al. Blood 96: 1425-1432 (2000), entirely incorporated by reference). Due to the incredible diversity of the antibody repertoire, mutations that lower affinity to known antibodies will typically lead to production of an another set of antibodies rather than abrogation of immunogenicity. However, in some cases it may be possible to decrease surface antigenicity by replacing hydrophobic and charged residues on the protein surface with polar neutral residues (see Meyer et. al. Protein Sci. 10: 491-503 (2001), entirely incorporated by reference).

An alternate approach is to disrupt T-cell activation. Removal of MHC-binding agretopes offers a much more tractable approach to immunogenicity reduction, as the diversity of MHC molecules comprises only 10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see for example WO 98/52976, WO 02/079232, and WO 00/3317, all entirely incorporated by reference.

While mutations in MHC-binding agretopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

There remains a need for novel C1q SF member proteins having reduced immunogenicity. Variants of C1q SF member with reduced immunogenicity could find use in the treatment of a number of C1q SF member responsive conditions.

### SUMMARY OF THE INVENTION

In accordance with the objects outlined above, the present invention provides novel C1q SF member proteins having reduced immunogenicity as compared to naturally occurring C1q SF member proteins. In an additional aspect, the present invention is directed to methods for engineering or designing less immunogenic proteins with C1q SF member activity for therapeutic use.

An aspect of the present invention are C1q SF member variants that show decreased binding affinity for one or more class II MHC alleles relative to a parent C1q SF member and which significantly maintain the activity of native naturally occurring C1q SF member.

In a further aspect, the invention provides recombinant nucleic acids encoding the variant C1q SF member proteins, expression vectors, and host cells.

In an additional aspect, the invention provides methods of producing a variant C1q SF member protein comprising culturing the host cells of the invention under conditions suitable for expression of the variant C1q SF member protein.

In a further aspect, the invention provides pharmaceutical compositions comprising a variant C1q SF member protein or nucleic acid of the invention and a pharmaceutical carrier.

In a further aspect, the invention provides methods for preventing or treating C1q SF member responsive disorders comprising administering a variant C1q SF member protein or nucleic acid of the invention to a patient.

In an additional aspect, the invention provides methods for screening the class II MHC haplotypes of potential patients in order to identify individuals who are particularly likely to raise an immune response to a wild type or variant C1q SF member therapeutic.

In accordance with the objects outlined above, the present invention provides C1q SF member variant proteins comprising amino acid sequences with at least one amino acid insertion, deletion, or substitution compared to the wild type C1q SF member proteins.

### BRIEF DESCRIPTION OF THE FIGURES

FIG. 1 shows a method for engineering less immunogenic C1q SF member derivatives.

FIG. 2 shows a schematic representation of a method for in vitro testing of the immunogenicity of C1q SF member peptides or proteins with IVV technology.

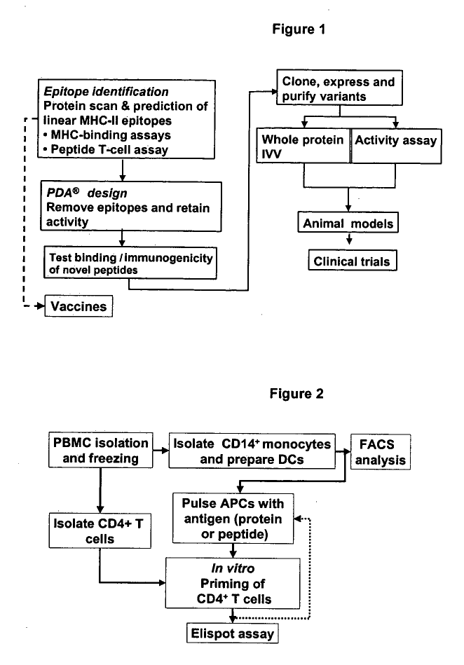

### DETAILED DESCRIPTION OF THE INVENTION

By “9-mer peptide frame” and grammatical equivalents herein is meant a linear sequence of nine amino acids that is located in a protein of interest. 9-mer frames may be analyzed for their propensity to bind one or more class II MHC alleles. By “allele” and grammatical equivalents herein is meant an alternative form of a gene. Specifically, in the context of class II MHC molecules, alleles comprise all naturally occurring sequence variants of DRA, DRB1, DRB3/4/5, DQA1, DQB1, DPA1, and DPB1 molecules. By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, a variant protein can be said to have “reduced immunogenicity” if it elicits neutralizing or non-neutralizing antibodies in lower titer or in fewer patients than the wild type protein. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A variant protein also can be said to have “reduced immunogenicity” if it shows decreased binding to one or more MHC alleles or if it induces T-cell activation in a decreased fraction of patients relative to the parent protein. In a preferred embodiment, the probability of T-cell activation is decreased by at least 5%, with at least 50% or 90% decreases being especially preferred. By “matrix method” and grammatical equivalents thereof herein is meant a method for calculating peptide-MHC affinity in which a matrix is used that contains a score for each possible residue at each position in the peptide, interacting with a given MHC allele. The binding score for a given peptide-MHC interaction is obtained by summing the matrix values for the amino acids observed at each position in the peptide. By “MHC-binding agretopes” and grammatical equivalents herein is meant peptides that are capable of binding to one or more class II MHC alleles with appropriate affinity to enable the formation of MHC-peptide-T-cell receptor complexes and subsequent T-cell activation. MHC-binding agretopes are linear peptide sequences that comprise at least approximately 9 residues. By “parent protein” as used herein is meant a protein that is subsequently modified to generate a variant protein. Said parent protein may be a wild-type or naturally occurring protein, or a variant or engineered version of a naturally occurring protein. “Parent protein” may refer to the protein itself, compositions that comprise the parent protein, or any amino acid sequence that encodes it. Accordingly, by “parent C1q SF member protein” as used herein is meant a C1q SF member protein that is modified to generate a variant C1q SF member protein. By “patient” herein is meant both humans and other animals, particularly mammals, and organisms. Thus the methods are applicable to both human therapy and veterinary applications. In the preferred embodiment the patient is a mammal, and in the most preferred embodiment the patient is human. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized. By “C1q SF member responsive disorders or conditions” and grammatical equivalents herein is meant diseases, disorders, and conditions that can benefit from treatment with C1q SF member. Examples of disorders that may benefit from treatment with C1q SF member or enhancers or inhibitors of C1q SF member include, but are not limited to, diseases associated with insulin resistance such as type 2 diabetes, obesity, impaired glucose tolerance (IGT), syndrome X, lipodystrophic disorders including HIV-associated lipodystrophy, anorexia, and other conditions associated with the regulation of glucose or lipid metabolism, cardiovascular diseases including atherosclerosis and coronary artery disease, and vascular restenosis following vascular intervention. C1q SF member may also be beneficial in promoting muscle growth, treating muscle wasting and other muscle-related disorders, preventing and treating liver diseases, and a variety of conditions associated with vascular damage including coronary angioplasty, carotid endarterectomy and stroke. By “treatment” herein is meant to include therapeutic treatment, as well as prophylactic, or suppressive measures for the disease or disorder. Thus, for example, successful administration of a variant C1q SF member protein prior to onset of the disease may result in treatment of the disease. As another example, successful administration of a variant C1q SF member protein after clinical manifestation of the disease to combat the symptoms of the disease comprises “treatment” of the disease. “Treatment” also encompasses administration of a variant C1q SF member protein after the appearance of the disease in order to eradicate the disease. Successful administration of an agent after onset and after clinical symptoms have developed, with possible abatement of clinical symptoms and perhaps amelioration of the disease, further comprises “treatment” of the disease. Those “in need of treatment” include mammals already having the disease or disorder, as well as those prone to having the disease or disorder, including those in which the disease or disorder is to be prevented. By “variant C1q SF member nucleic acids” and grammatical equivalents herein is meant nucleic acids that encode variant C1q SF member proteins. Due to the degeneracy of the genetic code, an extremely large number of nucleic acids may be made, all of which encode the variant C1q SF member proteins of the present invention, by simply modifying the sequence of one or more codons in a way that does not change the amino acid sequence of the variant C1q SF member. By “variant C1q SF member proteins” and grammatical equivalents thereof herein is meant non-naturally occurring C1q SF member proteins which differ from the wild type or parent C1q SF member protein by at least 1 amino acid insertion, deletion, or substitution. C1q SF member variants are characterized by the predetermined nature of the variation, a feature that sets them apart from naturally occurring allelic or interspecies variation of the C1q SF member protein sequence. The C1q SF member variants typically either exhibit biological activity that is comparable to naturally occurring C1q SF member or have been specifically engineered to have alternate biological properties. The variant C1q SF member proteins may contain insertions, deletions, and/or substitutions at the N-terminus, C-terminus, or internally. In a preferred embodiment, variant C1q SF member proteins have at least 1 residue that differs from the naturally occurring C1q SF member sequence, with at least 2, 3, 4, or 5 different residues being more preferred. Variant C1q SF member proteins may contain further modifications, for instance mutations that alter stability or solubility or which enable or prevent posttranslational modifications such as PEGylation or glycosylation. Variant C1q SF member proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild type sequence of adiponectin is SEQ_ID NO:1 and the wild type sequence of CTRP1 is SEQ_ID NO:2.

Identification of MHC-Binding Agretopes in C1q SF Member

MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naïve T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a 9-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in C1q SF member. Sequence-based information can be used to determine a binding score for a given peptide-MHC interaction (see for example Mallios, Bioinformatics 15: 432-439 (1999); Mallios, Bioinformatics 17: p 942-948 (2001); Sturniolo et. al. Nature Biotech. 17: 555-561 (1999), all entirely incorporated by reference). It is possible to use structure-based methods in which a given peptide is computationally placed in the peptide-binding groove of a given MHC molecule and the interaction energy is determined (for example, see WO 98/59244 and WO 02/069232, all entirely incorporated by reference). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187, entirely incorporated by reference).

In a preferred embodiment, MHC-binding propensity scores are calculated for each 9-residue frame along the C1q SF member sequence using a matrix method (see Sturniolo et. al., supra; Marshall et. al., J. Immunol. 154: 5927-5933 (1995), and Hammer et. al., J. Exp. Med. 180: 2353-2358 (1994), all entirely incorporated by reference). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding agretopes are identified as the 9-mer frames that bind to several class II MHC alleles. In an especially preferred embodiment, MHC-binding agretopes are predicted to bind at least 10 alleles at 5% threshold and/or at least 5 alleles at 1% threshold. Such 9-mer frames may be especially likely to elicit an immune response in many members of the human population.

In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet. 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003), all entirely incorporated by reference.

In a preferred embodiment, MHC binding agretopes are predicted for MHC heterodimers comprising highly prevalent MHC alleles. Class II MHC alleles that are present in at least 10% of the US population include but are not limited to: DPA1*0103, DPA1*0201, DPB1*0201, DPB1*0401, DPB1*0402, DQA1*0101, DQA1*0102, DQA1*0201, DQA1*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA1*0104, DPA1*0302, DPA1*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA1*0103, DQA1*0104, DQA1*0301, DQA1*0302, DQA1*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database.

MHC binding agretopes may also be predicted for MHC heterodimers comprising less prevalent alleles. Information about MHC alleles in humans and other species can be obtained, for example, from the IMGT/HLA sequence database.

In an especially preferred embodiment, an immunogenicity score is determined for each peptide, wherein said score depends on the fraction of the population with one or more MHC alleles that are hit at multiple thresholds. For example, the equation
Iscore=N(W₁P₁+W₃P₃+W₅P₅)
may be used, where P₁ is the percent of the population hit at 1%, P₃ is the percent of the population hit at 3%, P₅ is the percent of the population hit at 5%, each W is a weighting factor, and N is a normalization factor. In a preferred embodiment, W₁=10, W₃=5, W₅=2, and N is selected so that possible scores range from 0 to 100. In this embodiment, agretopes with Iscore greater than or equal to 10 are preferred and agretopes with Iscore greater than or equal to 25 are especially preferred.

In an additional preferred embodiment, MHC-binding agretopes are identified as the 9-mer frames that are located among “nested” agretopes, or overlapping 9-residue frames that are each predicted to bind a significant number of alleles. Such sequences may be especially likely to elicit an immune response.

Preferred MHC-binding agretopes are those agretopes that are predicted to bind, at a 3% threshold, to MHC alleles that are present in at least 5% of the population. Preferred MHC-binding agretopes in adiponectin include, but are not limited to, agretope 1: residues 109-117; agretope 2: residues 111-119; agretope 19: residues 122-130; agretope 6: residues 157-165; agretope 8: residues 160-168; agretope 9: residues 166-174; agretope 11: residues 175-183; agretope 12: residues 176-184; agretope 13: residues 202-210.

Especially preferred MHC-binding agretopes are those agretopes that are predicted to bind, at a 1% threshold, to MHC alleles that are present in at least 10% of the population. Especially preferred MHC-binding agretopes in adiponectin include, but are not limited to, agretope 1: residues 109-117; agretope 2: residues 111-119; agretope 3: residues 122-130; agretope 9: residues 166-174.

Preferred MHC-binding agretopes in CTRP1 include, but are not limited to, agretope 1: residues 150-158; agretope 3: residues 172-180; agretope 5: residues 185-193; agretope 11: residues 202-210; agretope 13: residues 209-217; agretope 14: residues 218-226; agretope 16: residues 230-238; agretope 17: residues 247-255; agretope 19: residues 267-275.

Especially preferred MHC-binding agretopes in CTRP1 include, but are not limited to, agretope 3: residues 172-180; agretope 14: residues 218-226; agretope 16: residues 230-238; agretope 17: residues 247-255.

Confirmation of MHC-Binding Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naïve T cells and antigen presenting cells from matched donors can be stimulated with a peptide containing an agretope of interest, and T-cell activation can be monitored. It is also possible to first stimulate T cells with the whole protein of interest, and then re-stimulate with peptides derived from the whole protein. If sera are available from patients who have raised an immune response to adiponectin, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Patient Genotype Analysis and Screening

HLA genotype is a major determinant of susceptibility to specific autoimmune diseases (see for example Nepom Clin. Immunol. Immunopathol. 67: S50-S55 (1993), entirely incorporated by reference) and infections (see for example Singh et. al. Emerg. Infect. Dis. 3: 41-49 (1997), entirely incorporated by reference). Furthermore, the set of MHC alleles present in an individual can affect the efficacy of some vaccines (see for example Cailat-Zucman et. al. Kidney Int. 53: 1626-1630 (1998) and Poland et. al. Vaccine 20: 430-438 (2001), all entirely incorporated by reference). HLA genotype may also confer susceptibility for an individual to elicit an unwanted immune response to an adiponectin therapeutic.

In a preferred embodiment, class II MHC alleles that are associated with increased or decreased susceptibility to elicit an immune response to adiponectin proteins are identified. For example, patients treated with adiponectin therapeutics may be tested for the presence of anti-adiponectin antibodies and genotyped for class II MHC. Alternatively, T-cell activation assays such as those described above may be conducted using cells derived from a number of genotyped donors. Alleles that confer susceptibility to adiponectin immunogenicity may be defined as those alleles that are significantly more common in those who elicit an immune response versus those who do not. Similarly, alleles that confer resistance to adiponectin immunogenicity may be defined as those that are significantly less common in those who do not elicit an immune response versus those that do. It is also possible to use purely computational techniques to identify which alleles are likely to recognize adiponectin therapeutics.

In one embodiment, the genotype association data is used to identify patients who are especially likely or especially unlikely to raise an immune response to an adiponectin therapeutic.

Design of Active, Less-Immunogenic Variants

In a preferred embodiment, the above-determined MHC-binding agretopes are replaced with alternate amino acid sequences to generate active variant adiponectin proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more 9-mer agretope identified above, one or more possible alternate 9-mer sequences are analyzed for immunogenicity as well as structural and functional compatibility. The preferred alternate 9-mer sequences are then defined as those sequences that have low predicted immunogenicity and a high probability of being structured and active. It is possible to consider only the subset of 9-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Substitution Matrices

In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see for example, Bowie et. al. Science 247: 1306-1310 (1990), Bowie and Sauer Proc. Nat. Acad. Sd. USA 86: 2152-2156 (1989), and Reidhaar-Olson and Sauer Proteins 7: 306-316 (1990), all entirely incorporated by reference). However, non-conservative mutations can destabilize protein structure and reduce activity (see for example, Lim et. al. Biochem. 31: 4324-4333 (1992), entirely incorporated by reference). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997), entirely incorporated by reference). The use of substitution matrices to design peptides with improved properties has been disclosed; see Adenot et al. J. Mol. Graph. Model. 17: 292-309 (1999), entirely incorporated by reference.

Substitution matrices include, but are not limited to, the BLOSUM matrices (Henikoff and Henikoff, Proc. Nat. Acad. Sci. USA 89: 10917 (1992), entirely incorporated by reference, the PAM matrices, the Dayhoff matrix, and the like. For a review of substitution matrices, see for example Henikoff Curr. Opin. Struct. Biol. 6: 353-360 (1996), entirely incorporated by reference. It is also possible to construct a substitution matrix based on an alignment of a given protein of interest and its homologs; see for example Henikoff and Henikoff Comput. Appl. Biosci. 12: 135-143 (1996), entirely incorporated by reference.

In a preferred embodiment, each of the substitution mutations that are considered has a BLOSUM 62 score of zero or higher. According to this metric, preferred substitutions include, but are not limited to:

TABLE 1
Conservative mutations

| Wild type | Preferred |
|---|---|
| residue | substitutions |
| A | C S T A G V |
| C | C A |
| D | S N D E Q |
| E | S N D E Q H R K |
| F | M I L F Y W |
| G | S A G N |
| H | N E Q H R Y |
| I | M I L V F |
| K | S N E Q R K |
| L | M I L V F |
| M | Q M I L V F |
| N | S T G N D E Q H R K |
| P | P |
| Q | S N D E Q H R K M |
| R | N E Q H R K |
| S | S T A G N D E Q K |
| T | T A M I L V |
| V | S T A N V |
| W | F Y W |
| Y | H F Y W |

In addition, it is preferred that the total BLOSUM 62 score of an alternate sequence for a nine residue MHC-binding agretope is decreased only modestly when compared to the BLOSUM 62 score of the wild type nine residue agretope. In a preferred embodiment, the score of the variant 9-mer is at least 50% of the wild type score, with at least 67%, 75%, 80% or 90% being especially preferred.

Alternatively, alternate sequences can be selected that minimize the absolute reduction in BLOSUM score; for example it is preferred that the score decrease for each 9-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine Substitutions

In an alternate embodiment, one or more alanine substitutions may be made, regardless of whether an alanine substitution is conservative or non-conservative. As is known in the art, incorporation of sufficient alanine substitutions may be used to disrupt intermolecular interactions.

In a preferred embodiment, variant 9-mers are selected such that residues that have been or can be identified as especially critical for maintaining the structure or function of adiponectin retain their wild type identity. In alternate embodiments, it may be desirable to produce variant adiponectin proteins that do not retain wild type activity. In such cases, residues that have been identified as critical for function may be specifically targeted for modification.

Adiponectin residues associated with diabetes and/or hypoadiponectinemia in humans include G84, G90, R92, Y111, R112 and 1164; C36 (C39 in mouse) participates in disulfide bonds and is believed to be critical for assembly of hexameric and high molecular weight (HMW) multimers; residues G84 and G90 have also been implicated in formation of HMW multimers; R112, 1164, and Y159 may affect trimer formation, with mutations at these positions resulting in impaired secretion from the cell. Hexameric and HMW isoforms have been reported to activate NF-κB pathways while trimers do not; oligomeric state may also affect the ability to activate AMP-activated protein kinase (see Waki et al. J. Biol. Chem. 278: 40352-40363 (2003), Tsao et al. J. Biol. Chem. 278: 50810-50817 (2003), and Kishida et al. Biochem. Biophys. Res. Commun. 306: 286-292 (2003) entirely incorporated by reference). The globular domain, but not the full length hexameric adiponectin enhances fatty acid oxidation in muscle and causes weight loss in mice (see Tomas et al. Proc. Natl. Acad. Sci. USA 99: 16309-16313 (2002), Fruebis et al. Proc. Natl. Acad. Sci. USA 98: 2005-2010 (2001) entirely incorporated by reference), and the higher order oligomeric forms have been reported to be less active in reducing glucose output (see Pajvani et al. J. Biol. Chem. 278: 9073-9085 (2003) entirely incorporated by reference). The HMW form of adiponectin has been implicated in suppression of endothelial cell apoptosis and in conferring its vascular-protective activities (see Kobayashi et al. Circ. Res. 94: e27-31 (2004) entirely incorporated by reference). Hydroxylation and glycosylation of lysines in the collagenous domain (mouse residues 68, 71, 80 and 104) have also been implicated in the insulin-sensitizing effect of full length adiponectin in mammalian cells (see Wang et al. J. Biol. Chem. 277: 19521-19529 (2002) entirely incorporated by reference).

Protein Design Methods

Protein design methods and MHC agretope identification methods may be used together to identify stable, active, and minimally immunogenic protein sequences (see WO03/006154, entirely incorporated by reference). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in Table 1 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

A wide variety of methods are known for generating and evaluating sequences. These include, but are not limited to, sequence profiling (Bowie and Eisenberg, Science 253(5016): 164-70, (1991)), residue pair potentials (Jones, Protein Science 3: 567-574, (1994)), and rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)), all entirely incorporated by reference.

Protein Design Automation® (PDA®) Technology

In an especially preferred embodiment, rational design of improved adiponectin variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all entirely incorporated by reference).

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 1050 sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

PDA® utilizes three-dimensional structural information. In a most preferred embodiment, the structure of adiponectin is determined using X-ray crystallography or NMR methods, which are well known in the art. The crystal structure of the mouse adiponectin globular trimer (amino acids 110-247) was solved to 2.1 Å resolution (see Shapiro and Scherer Curr. Biol. 8: 335-338 (1998) entirely incorporated by reference); the structure of human adiponectin may be derived using homology modeling methods known in the art.

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

Combining Immunogenicity Reduction strategies

In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. No. 11/004,590, filed Dec. 3, 2004, entirely incorporated by reference.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity.

In an additional preferred embodiment, a variant protein with reduced binding affinity for one or more class II MHC alleles is further modified by derivitization with PEG or another molecule. As is known in the art, PEG may sterically interfere with antibody binding or improve protein solubility, thereby reducing immunogenicity. In an especially preferred embodiment, rational PEGylation methods are used U.S. Ser. No. 10/956,352, filed Sep. 30, 2004, entirely incorporated by reference. In a preferred embodiment, PDA® technology and matrix method calculations are used to remove more than one MHC-binding agretope from a protein of interest.

Generating the Variants

Variant adiponectin proteins of the invention and nucleic acids encoding them may be produced using a number of methods known in the art.

In a preferred embodiment, nucleic acids encoding the adiponectin variants are prepared by total gene synthesis, or by site-directed mutagenesis of a nucleic acid encoding a parent adiponectin protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001), all entirely incorporated by reference).

In a preferred embodiment, adiponectin variants are cloned into an appropriate expression vector and expressed in E. coli (see McDonald, J. R., Ko, C., Mismer, D., Smith, D. J. and Collins, F. Biochim. Biophys. Acta 1090: 70-80 (1991), entirely incorporated by reference). In an alternate preferred embodiment, adiponectin variants are expressed in mammalian cells, yeast, baculovirus, or in vitro expression systems. A number of expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001), entirely incorporated by reference). The choice of codons, suitable expression vectors and suitable host cells will vary depending on a number of factors, and may be easily optimized as needed.

In a preferred embodiment, the adiponectin variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, an adiponectin variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed. (1994), entirely incorporated by reference. The degree of purification necessary will vary depending on the desired use, and in some instances no purification will be necessary.

Protocols for the expression and purification of adiponectin have been disclosed for bacteria (see Ouchi et al. Circulation 100: 2473-2476 (1999), Fruebis et al. Proc Natl Acad Sci USA 98: 2005-2010 (2001), Fruebis et al. Proc. Natl. Acad. Sci. USA 98: 2005-2010 (2001), Yamauchi et al. Nat Med 7: 941-946 (2001), Yamauchi et al. Nat. Med. 7: 941-946 (2001), Hu et al. Sheng Wu Hua Xue Yu Sheng Wu Wu Li Xue Bao (Shanghai) 35: 1023-1028 (2003), all entirely incorporated by reference), and mammalian systems (see Berg et al. Nat. Med. 7: 947-953 (2001), Tsao et al. J. Biol. Chem. 277: 29359-29362 (2002), all entirely incorporated by reference).

Assaying the Activity of the Variants

The variant adiponectin proteins of the invention may be tested for activity using any of a number of methods, including but not limited to those described herein. Adiponectin concentration may be measured using an enzyme-linked immuosorbent assay (ELISA) (see for example Arita et al. Biochem. Biophys. Res. Commun. 257: 79-83 (1999) entirely incorporated by reference). In vitro methods used to assay for processes related to atherosclerotic plaque formation include monocyte adhesion to endothelium, myeloid differentiation, macrophage cytokine production and phagocytosis (Ouchi et al. Circulation 100: 2473-2476 (1999) entirely incorporated by reference), lipid accumulation in cultured macrophages Ouchi et al. Circulation 103: 1057-1063 (2001) entirely incorporated by reference), and proliferation and migration of human aortic smooth muscle cells (see Arita et al. Circulation 105: 2893-2898 (2002), Waki et al. J. Biol. Chem. 278: 40352-40363 (2003) entirely incorporated by reference). In vitro methods to measure insulin sensitivity include assaying insulin-mediated suppression of glucose production in primary hepatocytes (see Berg et al. Nat. Med. 7: 947-953 (2001) entirely incorporated by reference). In vivo studies on insulin sensitivity and lipid metabolism have been performed using various mouse models including wild type mice on a high fat/sucrose diet, ob/ob (obese diabetic), NOD (non-obese diabetic) or streptozotocin-treated mice (see Berg et al. Nat. Med. 7: 947-953 (2001), Fruebis et al. Proc. Natl. Acad. Sci. USA 98: 2005-2010 (2001), Yamauchi et al. Nat. Med. 7: 941-946 (2001), all entirely incorporated by reference); measurements include weight loss, plasma glucose, free fatty acid and triglyceride levels, and triglyceride content in liver and muscle.

Determining the Immunogenicity of the Variants

In a preferred embodiment, the immunogenicity of the adiponectin variants is determined experimentally to confirm that the variants do have reduced or eliminated immunogenicity relative to the parent protein.

In a preferred embodiment, ex vivo T-cell activation assays are used to experimentally quantitate immunogenicity. In this method, antigen presenting cells and naïve T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000), entirely incorporated by reference). Other suitable T-cell assays include those disclosed in Meidenbauer, et al. Prostate 43, 88-100 (2000); Schultes, B. C and Whiteside, T. L., J. Immunol. Methods 279, 1-15 (2003); and Stickler, et al., J. Immunotherapy, 23, 654-660 (2000), all entirely incorporated by reference.

In a preferred embodiment, the PBMC donors used for the above-described T-cell activation assays will comprise class II MHC alleles that are common in patients requiring treatment for adiponectin responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to adiponectin responsive disorders.

In a preferred embodiment, the MHC haplotype of PBMC donors or patients that raise an immune response to the wild type or variant adiponectin are compared with the MHC haplotype of patients who do not raise a response. This data may be used to guide preclinical and clinical studies as well as aiding in identification of patients who will be especially likely to respond favorably or unfavorably to the adiponectin therapeutic.

In an alternate preferred embodiment, immunogenicity is measured in transgenic mouse systems. For example, mice expressing fully or partially human class II MHC molecules may be used.

In an alternate embodiment, immunogenicity is tested by administering the adiponectin variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see for example Sonderstrup et. al. Immunol. Rev. 172: 335-343 (1999) and Forsthuber et. al. J. Immunol. 167: 119-125 (2001), all entirely incorporated by reference).

Formulation and Administration to Patients

Once made, the variant C1q SF member proteins and nucleic acids of the invention find use in a number of applications. In a preferred embodiment, the variant C1q SF member proteins are administered to a patient to treat a C1q SF member responsive disorder.

The pharmaceutical compositions of the present invention comprise a variant C1q SF member protein in a form suitable for administration to a patient. In a preferred embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of pharmaceutical compositions may be administered. Moreover, the compositions may be administered in combination with other therapeutics.

The administration of the variant C1q SF member proteins of the present invention, preferably in the form of a sterile aqueous solution, may be done in a variety of ways, including, but not limited to, orally, subcutaneously, intravenously, intranasally, transdermally, intraperitoneally, intramuscularly, parenterally, intrapulmonary, vaginally, rectally, or intraocularly. In some instances, for example, the variant C1q SF member protein may be directly applied as a solution or spray. Depending upon the manner of introduction, the pharmaceutical composition may be formulated in a variety of ways. In a preferred embodiment, a therapeutically effective dose of a variant C1q SF member protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, the concentration of the therapeutically active variant C1q SF member protein in the formulation may vary from about 0.1 to about 100 weight %. In another preferred embodiment, the concentration of the variant C1q SF member protein is in the range of 0.003 to 1.0 molar, with dosages from 0.03, 0.05, 0.1, 0.2, and 0.3 millimoles per kilogram of body weight being preferred. As is known in the art, adjustments for variant C1q SF member protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

In an alternate embodiment, variant C1q SF member nucleic acids may be administered; i.e., “gene therapy” approaches may be used. In this embodiment, variant C1q SF member nucleic acids are introduced into cells in a patient in order to achieve in vivo synthesis of a therapeutically effective amount of variant C1q SF member protein. Variant C1q SF member nucleic acids may be introduced using a number of techniques, including but not limited to transfection with liposomes, viral (typically retroviral) vectors, and viral coat protein-liposome mediated transfection (Dzau et al., Trends in Biotechnology 11:205-210 (1993), entirely incorporated by reference). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), all entirely incorporated by reference. For review of gene marking and gene therapy protocols see Anderson et al., Science 256:808-813 (1992), entirely incorporated by reference.

### EXAMPLES

### Example 1

### Identification of MHC-Binding Agretopes in C1q SF Members

Matrix method calculations (Sturniolo, supra) were conducted using the parent C1q SF members sequences: Adiponectin (SEQ_ID_NO:1) and CTRP1 (SEQ_ID_NO:2).

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1104, DRB1*1301, DRB1*1302, DRB1*1501, and DRB1*1502.

Table 2. Predicted MHC-binding agretopes in C1q SF members. Iscore, the number of alleles, and the percent of the population hit at 1%, 3%, and 5% thresholds are shown. Especially preferred agretopes are predicted to affect at least 10% of the population, using a 1% threshold.

| | | | | | | | | | |
|---|---|---|---|---|---|---|---|---|---|
| Agretope | | | | 1% | 3% | 5% | 1% | 3% | 5% |
| number | Residues | Sequence | Iscore | hits | hits | hits | pop | pop | pop |

Table 2.A. Predicted MHC-binding agretopes in Adiponectin.

| | | | | | | | | | |
|---|---|---|---|---|---|---|---|---|---|
| Ag. A1 | 109-117 | YVYRSAFSV | 36.9 | 2 | 5 | 7 | 11% | 44% | 57% |
| Ag. A2 | 111-119 | YRSAFSVGL | 47.4 | 2 | 5 | 7 | 34% | 40% | 49% |
| Ag. A3 | 122-130 | YVTIPNMPI | 73.9 | 8 | 9 | 10 | 55% | 65% | 66% |
| Ag. A4 | 128-136 | MPIRFTKIF | 0.4 | 0 | 0 | 1 | 0% | 0% | 2% |
| Ag. A5 | 136-144 | FYNQQNHYD | 1.1 | 0 | 1 | 1 | 0% | 2% | 2% |
| Ag. A6 | 157-165 | LYYFAYHIT | 14.8 | 0 | 2 | 2 | 0% | 24% | 24% |
| Ag. A7 | 158-166 | YYFAYHITV | 6.1 | 0 | 0 | 1 | 0% | 0% | 25% |
| Ag. A8 | 160-168 | FAYHITVYM | 21.1 | 1 | 2 | 4 | 9% | 22% | 31% |
| Ag. A9 | 166-174 | VYMKDVKVS | 38.1 | 5 | 7 | 7 | 25% | 37% | 37% |
| Ag. A10 | 167-175 | YMKDVKVSL | 5.2 | 0 | 0 | 1 | 0% | 0% | 21% |
| Ag. A11 | 175-183 | LFKKDKAML | 3.1 | 0 | 1 | 1 | 0% | 5% | 5% |
| Ag. A12 | 176-184 | FKKDKAMLF | 15.8 | 0 | 1 | 2 | 0% | 21% | 32% |
| Ag. A13 | 202-210 | LLHLEVGDQ | 11.7 | 0 | 2 | 4 | 0% | 15% | 24% |

Table 2.B. Predicted MHC-binding agretopes in CTRP1.

| | | | | | | | | | |
|---|---|---|---|---|---|---|---|---|---|
| Ag. B1 | 150-158 | VGRKKPMHS | 16.5 | 0 | 4 | 7 | 0% | 20% | 37% |
| Ag. B2 | 171-179 | FVNLYDHFN | 0.4 | 0 | 0 | 1 | 0% | 0% | 2% |
| Ag. B3 | 172-180 | VNLYDHFNM | 36.1 | 1 | 3 | 6 | 23% | 32% | 42% |
| Ag. B4 | 178-186 | FNMFTGKFY | 4.2 | 0 | 1 | 2 | 0% | 2% | 14% |
| Ag. B5 | 185-193 | FYCYVPGLY | 3.1 | 0 | 1 | 1 | 0% | 5% | 5% |
| Ag. B6 | 186-194 | YCYVPGLYF | 6.1 | 0 | 0 | 1 | 0% | 0% | 25% |
| Ag. B7 | 188-196 | YVPGLYFFS | 1.2 | 0 | 1 | 1 | 0% | 2% | 2% |
| Ag. B8 | 192-200 | LYFFSLNVH | 7.6 | 0 | 1 | 3 | 0% | 2% | 28% |
| Ag. B9 | 193-201 | YFFSLNVHT | 3.4 | 0 | 0 | 1 | 0% | 0% | 14% |
| Ag. B10 | 194-202 | FFSLNVHTW | 2.8 | 0 | 0 | 1 | 0% | 0% | 11% |
| Ag. B11 | 202-210 | WNQKETYLH | 6.7 | 1 | 1 | 2 | 5% | 5% | 7% |
| Ag. B12 | 208-216 | YLHIMKNEE | 1.3 | 0 | 0 | 1 | 0% | 0% | 5% |
| Ag. B13 | 209-217 | LHIMKNEEE | 6.0 | 1 | 2 | 3 | 2% | 8% | 9% |
| Ag. B14 | 218-226 | VVILFAQVG | 44.5 | 2 | 9 | 12 | 17% | 52% | 61% |
| Ag. B15 | 220-228 | ILFAQVGDR | 5.2 | 0 | 0 | 1 | 0% | 0% | 21% |
| Ag. B16 | 230-238 | IMQSQSLML | 34.5 | 1 | 1 | 2 | 25% | 25% | 42% |
| Ag. B17 | 247-255 | WVRLYKGER | 26.1 | 1 | 3 | 5 | 11% | 23% | 42% |
| Ag. B18 | 248-256 | VRLYKGERE | 1.3 | 0 | 0 | 1 | 0% | 0% | 5% |
| Ag. B19 | 267-275 | YITFSGYLV | 14.6 | 1 | 2 | 3 | 2% | 14% | 34% |

Table 3. Predicted MHC-binding agretopes in C1q SF members. DRB1 alleles that are predicted to bind to each allele at 1%, 3%, 5% and 10% cutoffs are marked with “1”, “3”, “5” or “10” respectively.

TABLE 3

| Agretope | | | | | | | | | | | | | | | | | |
|---|---|---|---|---|---|---|---|---|---|---|---|---|---|---|---|---|---|
| number | 101 | 102 | 301 | 401 | 402 | 404 | 405 | 408 | 701 | 801 | 1101 | 1102 | 1104 | 1301 | 1302 | 1501 | 1502 |

A. Alleles predicted to bind MHC agretopes in Adiponectin.

| | | | | | | | | | | | | | | | | | |
|---|---|---|---|---|---|---|---|---|---|---|---|---|---|---|---|---|---|
| Ag. A1 | 3 | — | — | — | 5 | — | — | — | 3 | 3 | — | 10 | — | 10 | 1 | 5 | 1 |
| Ag. A2 | 1 | 3 | — | 5 | — | — | 3 | 5 | 1 | — | — | — | — | — | 10 | — | 3 |
| Ag. A3 | 1 | 10 | — | 1 | 5 | 1 | 1 | 1 | 1 | — | 1 | — | 10 | — | — | 3 | 1 |
| Ag. A4 | — | — | — | — | 5 | — | — | — | — | 10 | — | — | — | 10 | 10 | — | — |
| Ag. A5 | — | — | — | 10 | — | — | 3 | — | — | — | — | — | — | — | — | — | — |
| Ag. A6 | — | — | — | — | — | — | — | — | — | — | — | — | — | — | — | 3 | 3 |
| Ag. A7 | 10 | — | — | — | — | — | — | — | 5 | — | 10 | — | — | — | — | — | 10 |
| Ag. A8 | — | — | — | 3 | 5 | — | 10 | 10 | 10 | — | — | 10 | — | 5 | 1 | — | — |
| Ag. A9 | — | — | — | — | 1 | — | — | — | — | 1 | 3 | 1 | 3 | 1 | 1 | — | — |
| Ag. A10 | — | — | 5 | — | — | — | — | — | — | — | — | — | — | — | — | — | — |
| Ag. A11 | — | — | — | — | — | — | — | — | — | 3 | — | 10 | — | 10 | — | — | — |
| Ag. A12 | — | — | 3 | 5 | — | — | — | — | — | — | — | — | — | — | — | — | — |
| Ag. A13 | — | — | — | — | — | 5 | 10 | 10 | — | 5 | 3 | 10 | 3 | — | — | — | — |

B. Alleles predicted to bind MHC agretopes in CTRP1.

| | | | | | | | | | | | | | | | | | |
|---|---|---|---|---|---|---|---|---|---|---|---|---|---|---|---|---|---|
| Ag. B1 | — | — | 10 | — | 5 | — | — | — | — | 3 | 5 | 3 | 3 | 3 | 5 | — | — |
| Ag. B2 | — | — | — | — | — | — | 5 | — | — | — | — | — | — | — | — | — | — |
| Ag. B3 | — | — | — | — | — | — | — | — | — | 5 | — | 5 | — | 3 | 5 | 1 | 3 |
| Ag. B4 | 5 | — | — | — | — | — | — | — | — | — | — | — | — | — | 10 | 10 | 3 |
| Ag. B5 | — | — | — | — | — | — | — | — | — | 3 | — | — | — | — | — | — | — |
| Ag. B6 | 10 | — | — | — | — | — | — | — | 5 | — | — | — | — | — | — | — | 10 |
| Ag. B7 | — | — | — | — | — | — | — | — | — | — | — | — | — | — | — | 10 | 3 |
| Ag. B8 | — | — | — | — | — | 5 | 10 | 10 | — | — | — | 3 | — | — | — | 5 | 10 |
| Ag. B9 | 10 | — | — | 5 | — | — | 10 | — | 10 | — | — | — | — | — | — | — | — |
| Ag. B10 | — | — | — | 10 | — | — | 10 | 10 | — | 10 | 5 | — | — | — | — | — | — |
| Ag. B11 | — | — | — | — | 5 | — | 10 | 10 | — | 1 | — | — | — | — | — | — | — |
| Ag. B12 | — | — | — | — | — | — | 10 | — | — | 5 | — | — | — | — | — | — | — |
| Ag. B13 | — | — | — | 10 | — | 3 | 1 | 5 | — | — | — | — | — | — | — | — | — |
| Ag. B14 | 1 | 1 | 3 | 5 | 3 | 3 | 3 | 3 | — | 10 | 10 | 5 | 5 | 3 | 3 | 10 | — |
| Ag. B15 | — | — | 5 | — | — | — | — | — | — | — | — | — | — | — | — | — | — |
| Ag. B16 | — | 10 | — | 10 | — | — | — | — | 1 | — | — | — | — | — | — | 5 | 10 |
| Ag. B17 | — | — | 5 | — | — | — | — | — | — | 3 | 1 | — | 5 | 10 | 3 | — | — |
| Ag. B18 | — | — | — | — | — | — | — | — | — | 5 | — | — | — | — | — | 10 | 10 |
| Ag. B19 | 3 | 10 | — | — | — | — | — | — | 10 | — | — | — | — | — | — | 5 | 1 |

### Example 2

### Identification of Suitable Less Immunogenic Sequences for MHC-Binding Agretopes in C1q SF Members

MHC-binding agretopes that were predicted to bind alleles present in at least 10% of the US population, using a 1% threshold, were analyzed to identify suitable less immunogenic variants. At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are entirely incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 80% of the wild type score.

Table 4. Suitable less immunogenic variants of C1q SF members. B(wt) is the BLOSUM62 score of the wild type 9-mer, I(alt) is the percent of the US population containing one or more MHC alleles that are predicted to bind the alternate 9-mer at a 1% threshold and is 0 for all variants listed in Table 4, and B(alt) is the BLOSUM62 score of the alternate 9-mer.

| | | |
|---|---|---|
| | Variant | |
| Variant | sequence | B(alt) |

Table 4.A.i: Suitable less immunogenic variants of
Adiponectin Agretope A1 (residues 109-117;
YVYRSAFSV); B(wt) = 45.

| | | |
|---|---|---|
| Var:A1 | YTYRSAFSV | 42 |
| Var:A2 | YAYRSAFSV | 42 |
| Var:A3 | YMYRSAFSV | 42 |
| Var:A4 | YIYRSAFSV | 44 |
| Var:A5 | YLYRSAFSV | 42 |
| Var:A6 | YVHRSAFSV | 40 |
| Var:A7 | YVWRSAFSV | 40 |
| Var:A8 | YVYRSAYSV | 42 |
| Var:A9 | YVYRSGWSV | 36 |
| Var:A10 | YVYRSAWST | 37 |
| Var:A11 | YVYKSSFST | 36 |

Table 4.A.ii: Suitable less immunogenic variants
of Adiponectin Agretope A2 (residues 111-119;
YRSAFSVGL); B(wt) = 44.

| | | |
|---|---|---|
| Var:A12 | YESAFSVGL | 39 |
| Var:A13 | YRDAFSVGL | 40 |
| Var:A14 | YRSAFDVGL | 40 |
| Var:A15 | YRSAFEVGL | 40 |
| Var:A16 | YRSAFQVGL | 40 |
| Var:A17 | YRSAFKVGL | 40 |
| Var:A18 | YNTAFSVGL | 36 |
| Var:A19 | YNAAFSVGL | 36 |
| Var:A20 | YNSAFTVGL | 36 |
| Var:A21 | YNSAFNVGL | 36 |
| Var:A22 | YNSAFSTGL | 36 |
| Var:A23 | YNSAFSAGL | 36 |
| Var:A24 | YNSAFSVGM | 37 |
| Var:A25 | YNSAFSVGV | 36 |
| Var:A26 | YQEAFSVGL | 36 |
| Var:A27 | YQSGFSVGL | 36 |
| Var:A28 | YQSAFTVGL | 37 |
| Var:A29 | YQSAFNVGL | 37 |
| Var:A30 | YQSAFSTGL | 37 |
| Var:A31 | YQSAFSAGL | 37 |
| Var:A32 | YQSAFSVGM | 38 |
| Var:A33 | YQSAFSVGV | 37 |
| Var:A34 | YQSAFSVGF | 36 |
| Var:A35 | YHTAFSVGL | 36 |
| Var:A36 | YHAAFSVGL | 36 |
| Var:A37 | YHSAFTVGL | 36 |
| Var:A38 | YHSAFNVGL | 36 |
| Var:A39 | YHSAFSTGL | 36 |
| Var:A40 | YHSAFSAGL | 36 |
| Var:A41 | YHSAFSVGM | 37 |
| Var:A42 | YHSAFSVGV | 36 |
| Var:A43 | YKEAFSVGL | 37 |
| Var:A44 | YKSGFSVGL | 37 |
| Var:A45 | YKSAFTVGL | 38 |
| Var:A46 | YKSAFNVGL | 38 |
| Var:A47 | YKSAFSTGL | 38 |
| Var:A48 | YKSAFSAGL | 38 |
| Var:A49 | YKSAFSVGM | 39 |
| Var:A50 | YKSAFSVGV | 38 |
| Var:A51 | YKSAFSVGF | 37 |
| Var:A52 | YRTAFSVGF | 37 |
| Var:A53 | YRAAFSVGF | 37 |
| Var:A54 | YREGFSVGL | 36 |
| Var:A55 | YREAFTVGL | 37 |
| Var:A56 | YREAFGVGL | 36 |
| Var:A57 | YREAFNVGL | 37 |
| Var:A58 | YREAFSTGL | 37 |
| Var:A59 | YREAFSAGL | 37 |
| Var:A60 | YREAFSVGM | 38 |
| Var:A61 | YREAFSVGV | 37 |
| Var:A62 | YREAFSVGF | 36 |
| Var:A63 | YRQAFSVGF | 36 |
| Var:A64 | YRKAFSVGF | 36 |
| Var:A65 | YRSGFTVGL | 37 |
| Var:A66 | YRSGFNVGL | 37 |
| Var:A67 | YRSGFSTGL | 37 |
| Var:A68 | YRSGFSAGL | 37 |
| Var:A69 | YRSGFSVGM | 38 |
| Var:A70 | YRSGFSVGF | 36 |
| Var:A71 | YRSAFNAGL | 38 |
| Var:A72 | YRSAFNVGF | 37 |
| Var:A73 | YRSAFSTGF | 37 |
| Var:A74 | YRSAFSAGF | 37 |
| Var:A75 | YRTAFSAGM | 36 |
| Var:A76 | YRAAFSAGM | 36 |

Table 4.A.iii: Suitable less immunogenic variants
of Adiponectin Agretope A3 (residues 122-130;
YVTIPNMPI); B(wt) = 49.

| | | |
|---|---|---|
| Var:A77 | YTTIPNMPI | 46 |
| Var:A78 | YATIPNMPI | 46 |
| Var:A79 | YVTIPEMPI | 43 |
| Var:A80 | YMTIPDMPI | 41 |
| Var:A81 | YMTIPQMPI | 40 |
| Var:A82 | YMTIPNQPI | 42 |
| Var:A83 | YITIPDMPI | 43 |
| Var:A84 | YITIPQMPI | 42 |
| Var:A85 | YITIPNQPI | 44 |
| Var:A86 | YLTVPNMPI | 45 |
| Var:A87 | YLTIPDMPI | 41 |
| Var:A88 | YLTIPQMPI | 40 |
| Var:A89 | YLTIPHMPI | 41 |
| Var:A90 | YLTIPNQPI | 42 |
| Var:A91 | YLTIPNVPI | 42 |
| Var:A92 | YLTIPNMPF | 42 |
| Var:A93 | YVTLPDMPI | 42 |
| Var:A94 | YVTVPDMPI | 43 |
| Var:A95 | YVTIPDQPI | 40 |
| Var:A96 | YVTIPDIPI | 40 |
| Var:A97 | YVTIPDVPI | 40 |
| Var:A98 | YVTIPDFPI | 40 |
| Var:A99 | YVTIPDMPM | 41 |
| Var:A100 | YVTIPDMPL | 42 |
| Var:A101 | YVTIPDMPV | 43 |
| Var:A102 | YVTIPDMPF | 40 |
| Var:A103 | YVTIPQMPV | 42 |
| Var:A104 | YVTIPHMPF | 40 |
| Var:A105 | YMTLPNFPI | 40 |
| Var:A106 | YMTLPNMPF | 40 |
| Var:A107 | YMTVPHMPI | 40 |
| Var:A108 | YMTVPNVPI | 41 |
| Var:A109 | YMTVPNMPV | 44 |
| Var:A110 | YMTVPNMPF | 41 |
| Var:A111 | YMTIPHMPV | 40 |
| Var:A112 | YMTIPNIPV | 41 |
| Var:A113 | YMTIPNLPV | 42 |
| Var:A114 | YMTIPNVPV | 41 |
| Var:A115 | YMTIPNFPV | 41 |
| Var:A116 | YITMPHMPI | 40 |
| Var:A117 | YITLPHMPI | 41 |
| Var:A118 | YITLPNFPI | 42 |
| Var:A119 | YITLPNMPF | 42 |
| Var:A120 | YITVPHMPI | 42 |
| Var:A121 | YITVPNVPI | 43 |
| Var:A122 | YITVPNMPV | 46 |
| Var:A123 | YITVPNMPF | 43 |
| Var:A124 | YITIPGMPV | 41 |
| Var:A125 | YITIPHMPM | 40 |
| Var:A126 | YITIPHMPV | 42 |
| Var:A127 | YITIPNIPV | 43 |
| Var:A128 | YITIPNIPF | 40 |
| Var:A129 | YITIPNLPV | 44 |
| Var:A130 | YITIPNLPF | 41 |
| Var:A131 | YITIPNVPV | 43 |
| Var:A132 | YITIPNVPF | 40 |
| Var:A133 | YITIPNFPV | 43 |
| Var:A134 | YITIPNFPF | 40 |
| Var:A135 | YLTLPNIPI | 40 |
| Var:A136 | YLTLPNFPI | 40 |
| Var:A137 | YLTLPNMPL | 42 |
| Var:A138 | YLTLPNMPV | 43 |
| Var:A139 | YLTIPSMPV | 40 |
| Var:A140 | YLTIPNIPV | 41 |
| Var:A141 | YLTIPNLPV | 42 |
| Var:A142 | YLTIPNFPV | 41 |
| Var:A143 | YVTVPHMPM | 40 |
| Var:A144 | YVTVPHMPV | 42 |
| Var:A145 | YVTVPNQPV | 43 |
| Var:A146 | YVTVPNQPF | 40 |
| Var:A147 | YVTVPNVPV | 43 |
| Var:A148 | YVTVPNVPF | 40 |
| Var:A149 | YITLPSMPV | 40 |
| Var:A150 | YITLPNIPL | 40 |
| Var:A151 | YITLPNLPL | 41 |
| Var:A152 | YITLPNVPL | 40 |
| Var:A153 | YITVPNIPM | 40 |
| Var:A154 | YITVPNLPM | 41 |
| Var:A155 | YITVPNFPM | 40 |

Table 4.A.iv: Suitable less immunogenic variants
of Adiponectin Agretope A9 (residues 166-174;
VYMKDVKVS); B(wt) = 44.

| | | |
|---|---|---|
| Var:A156 | VYMSDVKVS | 39 |
| Var:A157 | VYMKDMKVS | 41 |
| Var:A158 | VYMKDVKVT | 41 |
| Var:A159 | VFQKDVKVS | 36 |
| Var:A160 | VWMRDVKVS | 36 |
| Var:A161 | VWMKDAKVS | 36 |
| Var:A162 | VWMKDIKVS | 38 |
| Var:A163 | VWMKDLKVS | 36 |
| Var:A164 | VWMKDVKVA | 36 |
| Var:A165 | VWMKDVKVN | 36 |
| Var:A166 | VYQEDVKVS | 36 |
| Var:A167 | VYQQDVKVS | 36 |
| Var:A168 | VYQRDVKVS | 37 |
| Var:A169 | VYQKDTKVS | 37 |
| Var:A170 | VYQKDAKVS | 37 |
| Var:A171 | VYQKDIKVS | 39 |
| Var:A172 | VYQKDLKVS | 37 |
| Var:A173 | VYQKDVEVS | 36 |
| Var:A174 | VYQKDVQVS | 36 |
| Var:A175 | VYQKDVKVA | 37 |
| Var:A176 | VYQKDVKVG | 36 |
| Var:A177 | VYQKDVKVN | 37 |
| Var:A178 | VYQKDVKVD | 36 |
| Var:A179 | VYQKDVKVE | 36 |
| Var:A180 | VYQKDVKVQ | 36 |
| Var:A181 | VYQKDVKVK | 36 |
| Var:A182 | VYLKDAKVS | 38 |
| Var:A183 | VYVEDVKVS | 36 |
| Var:A184 | VYVKDAKVS | 37 |
| Var:A185 | VYVKDLKVS | 37 |
| Var:A186 | VYVKDVEVS | 36 |
| Var:A187 | VYVKDVKVA | 37 |
| Var:A188 | VYVKDVKVG | 36 |
| Var:A189 | VYVKDVKVN | 37 |
| Var:A190 | VYVKDVKVK | 36 |
| Var:A191 | VYFEDVKVS | 36 |
| Var:A192 | VYFKDAKVS | 37 |
| Var:A193 | VYFKDLKVS | 37 |
| Var:A194 | VYFKDVEVS | 36 |
| Var:A195 | VYFKDVKVG | 36 |
| Var:A196 | VYFKDVKVK | 36 |
| Var:A197 | VYMNDAKVS | 36 |
| Var:A198 | VYMNDLKVS | 36 |
| Var:A199 | VYMNDVKVA | 36 |
| Var:A200 | VYMNDVKVN | 36 |
| Var:A201 | VYMEDAKVS | 37 |
| Var:A202 | VYMEDIKVS | 39 |
| Var:A203 | VYMEDLKVS | 37 |
| Var:A204 | VYMEDVKVA | 37 |
| Var:A205 | VYMEDVKVG | 36 |
| Var:A206 | VYMEDVKVN | 37 |
| Var:A207 | VYMEDVKVD | 36 |
| Var:A208 | VYMEDVKVE | 36 |
| Var:A209 | VYMEDVKVK | 36 |
| Var:A210 | VYMQDAKVS | 37 |
| Var:A211 | VYMQDVKVN | 37 |
| Var:A212 | VYMQDVKVD | 36 |
| Var:A213 | VYMQDVKVE | 36 |
| Var:A214 | VYMRDLKVS | 38 |
| Var:A215 | VYMRDVEVS | 37 |
| Var:A216 | VYMKDTEVS | 37 |
| Var:A217 | VYMKDASVS | 36 |
| Var:A218 | VYMKDAEVS | 37 |
| Var:A219 | VYMKDAKVA | 38 |
| Var:A220 | VYMKDAKVG | 37 |
| Var:A221 | VYMKDAKVN | 38 |
| Var:A222 | VYMKDAKVD | 37 |
| Var:A223 | VYMKDAKVK | 37 |
| Var:A224 | VYMKDISVS | 38 |
| Var:A225 | VYMKDIEVS | 39 |
| Var:A226 | VYMKDIKVG | 39 |
| Var:A227 | VYMKDIKVK | 39 |
| Var:A228 | VYMKDLSVS | 36 |
| Var:A229 | VYMKDLEVS | 37 |
| Var:A230 | VYMKDLQVS | 37 |
| Var:A231 | VYMKDLKVA | 38 |
| Var:A232 | VYMKDLKVG | 37 |
| Var:A233 | VYMKDLKVN | 38 |
| Var:A234 | VYMKDLKVK | 37 |
| Var:A235 | VYMKDVSVA | 36 |
| Var:A236 | VYMKDVEVA | 37 |
| Var:A237 | VYMKDVEVG | 36 |
| Var:A238 | VYMKDVEVN | 37 |
| Var:A239 | VYMKDVEVD | 36 |
| Var:A240 | VYMKDVEVE | 36 |
| Var:A241 | VYMKDVEVK | 36 |
| Var:A242 | VYLRDIKVS | 37 |
| Var:A243 | VYLKDIKVA | 37 |
| Var:A244 | VYLKDIKVN | 37 |
| Var:A245 | VYVRDIKVS | 36 |
| Var:A246 | VYFRDIKVS | 36 |
| Var:A247 | VYFKDIKVA | 36 |
| Var:A248 | VYFKDIKVN | 36 |
| Var:A249 | VYMQDIKVA | 36 |
| Var:A250 | VYMRDIKVA | 37 |
| Var:A251 | VYMKDIQVA | 36 |
| Var:A252 | VYMKDIQVN | 36 |

Table 4.B.1. Suitable less immunogenic variants of
CTRPI agretope B3 (residues 172-180,
VNLYDHFNM); B(wt) = 52.

| | | |
|---|---|---|
| Var:B1 | VSLYDHFNM | 47 |
| Var:B2 | VTLYDHFNM | 46 |
| Var:B3 | VGLYDHFNM | 46 |
| Var:B4 | VDLYDHFNM | 47 |
| Var:B5 | VELYDHFNM | 46 |
| Var:B6 | VNVYDHFNM | 49 |
| Var:B7 | VNFYDHFNM | 48 |
| Var:B8 | VNLWDHFNM | 47 |
| Var:B9 | VNLYDEFNM | 44 |
| Var:B10 | VNLYDQFNM | 44 |
| Var:B11 | VNLYDHYNM | 49 |
| Var:B12 | VNLYDHWNM | 47 |
| Var:B13 | VNLYDHFNQ | 48 |
| Var:B14 | VNLYDHFNF | 48 |
| Var:B15 | VNLHDHFNL | 44 |
| Var:B16 | VNLHDHFNV | 43 |

Table 4.B.ii. Suitable less immunogenic variants
of CTRP1 agretope B14 (residues 218-226,
VVILFAQVG); B (wt) = 41.

| | | |
|---|---|---|
| Var:B17 | VTILFAQVG | 38 |
| Var:B18 | VAILFAQVG | 38 |
| Var:B19 | VVILFADVG | 36 |
| Var:B20 | VMLLFAQVG | 36 |
| Var:B21 | VMVLFAQVG | 37 |
| Var:B22 | VMFLFAQVG | 34 |
| Var:B23 | VMIVFAQVG | 35 |
| Var:B24 | VMIFFAQVG | 34 |
| Var:B25 | VMILFSQVG | 35 |
| Var:B26 | VMILFVQVG | 34 |
| Var:B27 | VMILFAEVG | 35 |
| Var:B28 | VILLFAQVG | 38 |
| Var:B29 | VIVLFAQVG | 39 |
| Var:B30 | VIFLFAQVG | 36 |
| Var:B31 | VIIVFAQVG | 37 |
| Var:B32 | VIIFFAQVG | 36 |
| Var:B33 | VIILFSQVG | 37 |
| Var:B34 | VIILFVQVG | 36 |
| Var:B35 | VIILFAEVG | 37 |
| Var:B36 | VLLLFAQVG | 36 |
| Var:B37 | VLVLFAQVG | 37 |
| Var:B38 | VLFLFAQVG | 34 |
| Var:B39 | VLIIFAQVG | 36 |
| Var:B40 | VLIVFAQVG | 35 |
| Var:B41 | VLIFFAQVG | 34 |
| Var:B42 | VLILFSQVG | 35 |
| Var:B43 | VLILFVQVG | 34 |
| Var:B44 | VLILFASVG | 33 |
| Var:B45 | VLILFAEVG | 35 |
| Var:B46 | VLILFAKVG | 34 |
| Var:B47 | VVLVFAQVG | 36 |
| Var:B48 | VVVVFAQVG | 37 |
| Var:B49 | VVVFFAQVG | 36 |
| Var:B50 | VVVLFSQVG | 37 |
| Var:B51 | VVVLFVQVG | 36 |
| Var:B52 | VVVLFAEVG | 37 |
| Var:B53 | VVFVFAQVG | 34 |
| Var:B54 | VVFFFAQVG | 33 |
| Var:B55 | VVFLFAEVG | 34 |
| Var:B56 | VVIVFAEVG | 35 |
| Var:B57 | VVIFFAEVG | 34 |
| Var:B58 | VMMIFAQVG | 33 |
| Var:B59 | VIMIFAQVG | 35 |
| Var:B60 | VIMLFAKVG | 33 |
| Var:B61 | VIIMFAKVG | 34 |
| Var:B62 | VIIIFGQVG | 34 |
| Var:B63 | VIIIFASVG | 33 |
| Var:B64 | VIIIFANVG | 33 |
| Var:B65 | VIIIFAHVG | 33 |
| Var:B66 | VIIIFARVG | 34 |
| Var:B67 | VIIIFAKVG | 34 |
| Var:B68 | VIIIFAMVG | 33 |
| Var:B69 | VVLIFAEVG | 34 |
| Var:B70 | VVLLFSEVG | 33 |
| Var:B71 | VVVMFAKVG | 34 |
| Var:B72 | VVVIFGQVG | 34 |
| Var:B73 | VVVIFASVG | 33 |
| Var:B74 | VVVIFANVG | 33 |
| Var:B75 | VVVIFAHVG | 33 |
| Var:B76 | VVVIFARVG | 34 |
| Var:B77 | VVVIFAKVG | 34 |
| Var:B78 | VVVIFAMVG | 33 |

Table 4.B.iii. Suitable less immunogenic variants
of CTRP1 agretope 816 (residues 230-238,
IMQSQSLML); B(wt) = 40.

| | | |
|---|---|---|
| Var:B79 | IMDSQSLML | 35 |
| Var:B80 | IMQAQSLML | 37 |
| Var:B81 | IMQGQSLML | 36 |
| Var:B82 | IMQNQSLML | 37 |
| Var:B83 | IMQDQSLML | 36 |
| Var:B84 | IMQEQSLML | 36 |
| Var:B85 | IMQQQSLML | 36 |
| Var:B86 | IMQKQSLML | 36 |
| Var:B87 | IMQSQDLML | 36 |
| Var:B88 | IMQSQELML | 36 |
| Var:B89 | IMQSQQLML | 36 |
| Var:B90 | IMQSQKLML | 36 |
| Var:B91 | IMQSQSLMM | 38 |
| Var:B92 | IMQSQSLMV | 37 |
| Var:B93 | IMQSQSLMF | 36 |
| Var:B94 | ILESQSLML | 34 |
| Var:B95 | ILQSQGLML | 33 |
| Var:B96 | ILQSQNLML | 34 |
| Var:B97 | IFESQSLML | 33 |
| Var:B98 | IFQSQGLML | 32 |
| Var:B99 | IFQSQNLML | 33 |
| Var:B100 | IMETQSLML | 34 |
| Var:B101 | IMESQTLML | 34 |
| Var:B102 | IMESQALML | 34 |
| Var:B103 | IMESQGLML | 33 |
| Var:B104 | IMESQNLML | 34 |
| Var:B105 | IMESQSMML | 35 |
| Var:B106 | IMESQSVML | 34 |
| Var:B107 | IMESQSFML | 33 |
| Var:B108 | IMQTQGLML | 33 |
| Var:B109 | IMQTQNLML | 34 |
| Var:B110 | IMQSQTVML | 34 |
| Var:B111 | IMQSQGMML | 34 |
| Var:B112 | IMQSQGVML | 33 |
| Var:B113 | IMQSQGFML | 32 |
| Var:B114 | IMQSQNMML | 35 |
| Var:B115 | IMQSQNVML | 34 |
| Var:B116 | IMQSQNFML | 33 |

Table 4.B.iv. Suitable less immunogenic variants
of CTRPI agretope B17 (residues 247-255,
WVRLYKGER); B(wt) = 52.

| | | |
|---|---|---|
| Var:B117 | WTRLYKGER | 49 |
| Var:B118 | WARLYKGER | 49 |
| Var:B119 | WMRLYKGER | 49 |
| Var:B120 | WIRLYKGER | 51 |
| Var:B121 | WLRLYKGER | 49 |
| Var:B122 | WVELYKGER | 47 |
| Var:B123 | WVQLYKGER | 48 |
| Var:B124 | WVHLYKGER | 47 |
| Var:B125 | WVKLYKGER | 49 |
| Var:B126 | WVRVYKGER | 49 |
| Var:B127 | WVRLYSGER | 47 |
| Var:B128 | WVRLYNGER | 47 |
| Var:B129 | WVRLYEGER | 48 |
| Var:B130 | WVRLYQGER | 48 |
| Var:B131 | WVNIYKGER | 45 |
| Var:B132 | WVNFYKGER | 43 |
| Var:B133 | WVNLYRGER | 44 |
| Var:B134 | WVRMYRGER | 47 |
| Var:B135 | WVRIYRGER | 47 |
| Var:B136 | WVRIYKSER | 44 |
| Var:B137 | WVRLYRSER | 43 |

### Example 3

### Identification of Suitable Less Immunogenic Sequences for MHC-Binding Agretopes as Determined by PDA® Technology

Table 5. Each position in the agretopes of interest was analyzed to identify a subset of amino acid substitutions that are potentially compatible with maintaining the structure and function of the protein. PDA® technology calculations were run for each position of each 9-mer agretope and compatible amino acids for each position were saved. In these calculations, side-chains within 5 Angstroms of the position of interest were permitted to change conformation but not amino acid identity. The variant agretopes were then analyzed for immunogenicity. The PDA® energies and Iscore values for the wild-type 9-mer agretope were compared to the variants and the subset of variant sequences with lower predicted immunogenicity and PDA® energies within 5.0 kcal/mol of the wild-type were noted. In the tables below, E(PDA) is the energy determined using PDA® technology calculations compared against the wild-type, Iscore: Anchor is the Iscore for the agretope, and Iscore: Overlap is the sum of the Iscores for all of the overlapping agretopes.

TABLE 5

| | | | |
|---|---|---|---|
| | | Iscore | Iscore |
| Var. | E(PDA) | Anchor | Overlap |

A.i. Less immunogenic variants of Adiponectin agretope A1.

| | | | |
|---|---|---|---|
| wt | 0.00 | 36.9 | 47.4 |
| Y109A | 2.18 | 0.0 | 47.4 |
| Y109D | 2.29 | 0.0 | 47.4 |
| Y109E | 1.87 | 0.0 | 47.4 |
| Y109G | 2.07 | 0.0 | 47.4 |
| Y109H | 1.18 | 0.0 | 47.4 |
| Y109I | 4.12 | 29.4 | 47.4 |
| Y109K | 1.90 | 0.0 | 47.4 |
| Y109L | 2.71 | 29.4 | 47.4 |
| Y109M | 3.47 | 29.4 | 47.4 |
| Y109N | 1.81 | 0.0 | 47.4 |
| Y109P | 2.87 | 0.0 | 47.4 |
| Y109Q | 1.01 | 0.0 | 47.4 |
| Y109R | 1.86 | 0.0 | 47.4 |
| Y109S | 2.58 | 0.0 | 47.4 |
| Y109T | 2.41 | 0.0 | 47.4 |
| Y109V | 3.02 | 29.4 | 47.4 |
| V110A | −1.07 | 0.0 | 47.4 |
| V110D | −1.17 | 0.0 | 47.4 |
| V110E | −1.01 | 0.0 | 47.4 |
| V110F | −0.20 | 3.8 | 47.4 |
| V110G | 0.04 | 0.0 | 47.4 |
| V110H | −0.21 | 3.8 | 47.4 |
| V110I | 1.17 | 12.8 | 47.4 |
| V110K | −1.55 | 12.8 | 47.4 |
| V110L | −1.31 | 3.8 | 47.4 |
| V110M | 0.24 | 12.8 | 47.4 |
| V110N | −1.38 | 3.8 | 47.4 |
| V110P | −0.34 | 0.0 | 47.4 |
| V110Q | −1.69 | 12.8 | 47.4 |
| V110S | −0.60 | 0.0 | 47.4 |
| V110T | −0.67 | 0.0 | 47.4 |
| V110W | 2.12 | 0.0 | 47.4 |
| V110Y | −0.27 | 3.8 | 47.4 |
| Y111A | 2.41 | 14.5 | 0.0 |
| Y111D | 2.65 | 0.0 | 0.0 |
| Y111E | 3.44 | 0.0 | 0.0 |
| Y111G | 4.20 | 16.4 | 0.0 |
| Y111H | 1.49 | 16.4 | 0.0 |
| Y111K | 2.72 | 14.5 | 0.0 |
| Y111N | 1.02 | 24.4 | 0.0 |
| Y111P | 0.30 | 16.4 | 0.0 |
| Y111Q | 3.59 | 14.5 | 0.0 |
| Y111R | 2.71 | 33.8 | 0.0 |
| Y111S | 2.55 | 16.4 | 0.0 |
| Y111T | 3.14 | 14.5 | 0.0 |
| Y111V | 4.65 | 24.4 | 44.8 |
| Y111W | 2.47 | 14.5 | 47.4 |
| R112D | 1.71 | 35.3 | 0.0 |
| R112G | 3.32 | 31.2 | 20.9 |
| R112K | −0.45 | 36.3 | 36.1 |
| A114G | 4.02 | 27.8 | 43.4 |
| F115H | 4.27 | 16.1 | 47.4 |
| F115M | 4.88 | 36.0 | 47.4 |
| F115Y | 1.05 | 27.6 | 47.4 |
| V117A | 4.70 | 16.2 | 32.8 |
| V117S | 3.18 | 32.7 | 45.9 |
| V117T | 2.44 | 15.1 | 46.6 |

A.ii. Less immunogenic variants of Adiponectin agretope A2.

| | | | |
|---|---|---|---|
| wt | 0.00 | 47.4 | 36.9 |
| Y111G | 4.20 | 0.0 | 16.4 |
| Y111H | 1.49 | 0.0 | 16.4 |
| Y111K | 2.72 | 0.0 | 14.5 |
| Y111N | 1.02 | 0.0 | 24.4 |
| Y111P | 0.30 | 0.0 | 16.4 |
| Y111Q | 3.59 | 0.0 | 14.5 |
| Y111R | 2.71 | 0.0 | 33.8 |
| Y111S | 2.55 | 0.0 | 16.4 |
| Y111T | 3.14 | 0.0 | 14.5 |
| Y111V | 4.65 | 44.8 | 24.4 |
| R112D | 1.71 | 0.0 | 35.3 |
| R112G | 3.32 | 20.9 | 31.2 |
| R112K | −0.45 | 36.1 | 36.3 |
| S113A | −0.86 | 46.6 | 36.9 |
| Y111A | 2.41 | 0.0 | 14.5 |
| Y111D | 2.65 | 0.0 | 0.0 |
| Y111E | 3.44 | 0.0 | 0.0 |
| A114G | 4.02 | 43.4 | 27.8 |
| V117A | 4.70 | 32.8 | 16.2 |
| V117S | 3.18 | 45.9 | 32.7 |
| V117T | 2.44 | 46.6 | 15.1 |
| L119A | 3.52 | 26.5 | 36.9 |
| L119D | 4.20 | 2.2 | 36.9 |
| L119E | 2.88 | 2.2 | 36.9 |
| L119K | 2.73 | 11.8 | 36.9 |
| L119N | 3.70 | 8.8 | 36.9 |
| L119Q | 2.65 | 28.4 | 36.9 |
| L119R | 3.28 | 12.4 | 36.9 |
| L119S | 3.24 | 40.9 | 36.9 |
| L119T | 3.20 | 23.8 | 36.9 |

A.iii. Less immunogenic variants of Adiponectin agretope A3.

| | | | |
|---|---|---|---|
| wt | 0.00 | 74.0 | 0.4 |
| Y122D | −0.24 | 0.0 | 0.4 |
| Y122E | −0.42 | 0.0 | 0.4 |
| Y122H | −0.34 | 0.0 | 0.4 |
| Y122K | −0.62 | 0.0 | 0.4 |
| Y122Q | −0.02 | 0.0 | 0.4 |
| Y122R | −0.04 | 0.0 | 0.4 |
| V123A | 3.57 | 19.4 | 0.4 |
| V123E | 1.75 | 23.2 | 0.4 |
| V123I | 0.14 | 57.7 | 0.4 |
| V123N | 4.79 | 28.4 | 0.4 |
| V123P | 1.06 | 6.7 | 0.4 |
| V123Q | 3.91 | 59.9 | 0.4 |
| V123S | 4.53 | 9.6 | 0.4 |
| V123T | 2.06 | 19.4 | 0.4 |
| T124D | 2.89 | 28.4 | 0.4 |
| T124E | 2.13 | 43.4 | 0.4 |
| I125D | −1.30 | 37.7 | 0.4 |
| I125E | −1.53 | 29.9 | 0.4 |
| I125F | 0.16 | 70.5 | 0.4 |
| I125G | 0.48 | 31.0 | 0.4 |
| I125H | −0.48 | 68.0 | 0.4 |
| I125K | −0.45 | 14.6 | 0.4 |
| I125L | −0.53 | 65.5 | 0.4 |
| I125N | −1.13 | 45.0 | 0.4 |
| I125P | 3.74 | 23.9 | 0.4 |
| I125Q | −1.14 | 35.9 | 0.4 |
| I125R | −0.34 | 31.0 | 0.4 |
| I125S | −1.06 | 51.5 | 0.4 |
| I125T | −0.76 | 49.2 | 0.4 |
| I125V | −0.94 | 57.5 | 0.4 |
| I125W | −0.16 | 38.4 | 0.4 |
| I125Y | −1.27 | 47.2 | 0.4 |
| N127D | 0.45 | 23.9 | 0.4 |
| N127G | 0.60 | 57.3 | 0.4 |
| N127K | 0.38 | 63.7 | 0.4 |
| M128A | −0.77 | 48.1 | 0.0 |
| M128D | −0.36 | 18.6 | 0.0 |
| M128E | −0.49 | 50.7 | 0.0 |
| M128G | 0.21 | 21.3 | 0.0 |
| M128H | −0.68 | 60.8 | 0.0 |
| M128I | −0.10 | 69.5 | 0.4 |
| M128K | 0.24 | 36.5 | 0.0 |
| M128L | −0.61 | 73.2 | 0.4 |
| M128N | −0.62 | 63.4 | 0.0 |
| M128Q | −0.78 | 58.9 | 0.0 |
| M128R | 0.57 | 51.2 | 0.0 |
| M128S | −0.72 | 40.4 | 0.0 |
| M128T | −1.02 | 57.8 | 0.0 |
| M128V | −1.23 | 59.3 | 0.4 |
| I130A | 4.58 | 48.6 | 0.0 |
| I130D | 4.53 | 15.8 | 0.0 |
| I130E | 3.95 | 16.6 | 0.0 |
| I130L | −0.60 | 63.6 | 0.4 |
| I130M | 4.35 | 50.6 | 0.4 |
| I130N | 3.01 | 26.5 | 0.0 |
| I130Q | 3.18 | 41.1 | 0.0 |
| I130V | 1.08 | 51.4 | 0.0 |

A.iv. Less immunogenic variants of Adiponectin agretope A9.

| | | | |
|---|---|---|---|
| wt | 0.00 | 38.0 | 32.4 |
| V166A | 4.40 | 0.0 | 29.1 |
| V166S | 4.36 | 0.0 | 26.1 |
| V166T | 3.90 | 0.0 | 19.2 |
| Y167A | 2.28 | 23.7 | 28.1 |
| Y167D | 4.03 | 3.6 | 27.2 |
| Y167E | 2.81 | 23.7 | 28.4 |
| Y167F | −1.35 | 35.0 | 32.4 |
| Y167G | 3.57 | 34.2 | 27.2 |
| Y167H | 0.35 | 35.0 | 28.4 |
| Y167W | 4.96 | 23.7 | 32.4 |
| M168A | −1.42 | 16.0 | 26.0 |
| M168G | −0.85 | 16.0 | 19.4 |
| K169A | −1.29 | 36.2 | 32.4 |
| K169E | −0.88 | 20.3 | 27.2 |
| K169T | 1.61 | 16.7 | 32.4 |
| K169W | 0.81 | 34.5 | 32.4 |
| V171A | −1.04 | 16.5 | 32.4 |
| V171G | 1.25 | 4.7 | 32.4 |
| V171N | 3.19 | 10.4 | 32.4 |
| V171S | −0.80 | 17.6 | 32.4 |
| V171T | −3.38 | 35.0 | 32.4 |
| K172D | 4.99 | 10.3 | 27.2 |
| K172E | 3.20 | 24.8 | 27.2 |
| K172G | 2.45 | 22.8 | 27.2 |
| K172Q | 1.24 | 36.3 | 27.2 |
| K172R | 0.75 | 35.0 | 32.4 |
| S174A | −1.39 | 26.4 | 32.4 |
| S174G | 0.51 | 24.0 | 32.4 |

B.i. Less immunogenic variants of CTRP1 agretope B3.

| | | | |
|---|---|---|---|
| wt | 0.00 | 36.1 | 4.6 |
| V172A | −3.17 | 0.0 | 4.2 |
| V172D | 2.37 | 0.0 | 4.2 |
| V172G | −1.77 | 0.0 | 4.2 |
| V172N | 1.77 | 0.0 | 4.2 |
| V172S | −3.40 | 0.0 | 4.2 |
| V172T | −3.46 | 0.0 | 4.2 |
| L174A | −2.00 | 14.3 | 4.2 |
| L174D | −0.77 | 0.0 | 4.2 |
| L174E | −1.45 | 0.0 | 4.2 |
| L174G | −1.41 | 14.3 | 4.2 |
| L174H | −2.92 | 14.3 | 4.2 |
| L174K | −3.13 | 14.3 | 4.2 |
| L174N | −2.51 | 16.8 | 4.2 |
| L174P | −2.47 | 16.2 | 4.2 |
| L174Q | −3.76 | 14.3 | 4.2 |
| L174R | −3.19 | 19.7 | 4.2 |
| L174S | −1.74 | 14.3 | 4.2 |
| L174T | −2.24 | 14.3 | 4.2 |
| L174V | −2.45 | 16.8 | 4.2 |
| L174W | −0.14 | 14.3 | 4.2 |
| Y175A | −5.66 | 2.6 | 4.6 |
| Y175D | −2.89 | 26.2 | 4.6 |
| Y175E | −4.66 | 0.0 | 4.6 |
| Y175G | −4.73 | 5.2 | 4.6 |
| Y175I | 4.29 | 10.0 | 4.6 |
| Y175K | −6.05 | 12.7 | 4.6 |
| Y175L | −3.94 | 8.8 | 4.6 |
| Y175M | −4.89 | 16.2 | 4.6 |
| Y175N | −4.24 | 11.1 | 4.6 |
| Y175P | −8.97 | 0.0 | 4.6 |
| Y175T | −6.65 | 15.2 | 4.6 |
| H177D | 2.27 | 28.7 | 4.2 |
| H177E | 2.47 | 14.8 | 4.2 |
| H177G | 1.04 | 31.6 | 4.2 |
| H177Q | −4.02 | 21.2 | 4.6 |
| F178H | 4.97 | 20.1 | 0.4 |
| F178K | 4.92 | 3.7 | 0.4 |
| F178Y | 1.16 | 14.3 | 4.6 |
| M180A | −1.74 | 29.0 | 0.4 |
| M180D | 0.64 | 1.3 | 0.4 |
| M180E | 0.76 | 3.1 | 0.4 |
| M180F | −0.17 | 21.2 | 0.9 |
| M180G | −0.66 | 16.2 | 0.4 |
| M180H | −0.54 | 8.7 | 0.4 |
| M180K | −0.17 | 0.4 | 0.4 |
| M180L | 0.72 | 28.7 | 0.9 |
| M180N | −0.72 | 6.5 | 0.4 |
| M180P | −2.82 | 5.6 | 0.4 |
| M180Q | −0.12 | 4.1 | 0.4 |
| M180R | −1.15 | 7.5 | 1.4 |
| M180S | −1.18 | 22.7 | 0.4 |
| M180T | −1.58 | 14.8 | 0.4 |
| M180V | 0.58 | 28.7 | 0.4 |
| M180W | 0.87 | 5.6 | 0.4 |
| M180Y | −0.65 | 14.3 | 0.9 |

B.ii. Less immunogenic variants of CTRP1 agretope B14.

| | | | |
|---|---|---|---|
| wt | 0.00 | 44.5 | 5.2 |
| V218A | 0.76 | 0.0 | 5.2 |
| V218G | −3.23 | 0.0 | 5.2 |
| V218N | 3.63 | 0.0 | 5.2 |
| V218S | −0.65 | 0.0 | 5.2 |
| V218T | −1.01 | 0.0 | 5.2 |
| V219A | −6.10 | 3.1 | 5.2 |
| V219D | −3.41 | 0.0 | 5.2 |
| V219E | −1.07 | 5.9 | 5.2 |
| V219G | −5.86 | 10.3 | 5.2 |
| V219H | 0.32 | 13.3 | 5.2 |
| V219I | 0.40 | 14.5 | 5.2 |
| V219K | 0.94 | 14.5 | 5.2 |
| V219L | 2.84 | 14.5 | 5.2 |
| V219M | −2.09 | 14.5 | 5.2 |
| V219N | −5.87 | 13.3 | 5.2 |
| V219Q | 4.91 | 16.9 | 5.2 |
| V219S | −5.81 | 1.2 | 5.2 |
| V219T | −6.13 | 3.1 | 5.2 |
| I220A | −13.35 | 10.3 | 0.0 |
| I220D | −12.01 | 0.0 | 0.0 |
| I220G | −10.62 | 14.5 | 0.0 |
| I220L | −3.55 | 32.9 | 5.2 |
| I220N | −12.73 | 14.5 | 0.0 |
| I220S | −15.07 | 14.5 | 0.0 |
| I220T | −6.67 | 10.3 | 0.0 |
| I220V | −4.25 | 14.5 | 5.2 |
| L221A | −2.75 | 31.1 | 0.0 |
| L221D | −2.90 | 32.9 | 0.0 |
| L221E | −3.21 | 20.5 | 0.0 |
| L221F | −3.12 | 43.6 | 5.2 |
| L221G | −1.46 | 21.8 | 0.0 |
| L221I | −1.42 | 37.6 | 5.2 |
| L221K | −3.88 | 24.8 | 5.2 |
| L221N | −3.26 | 32.3 | 5.2 |
| L221P | 4.44 | 0.4 | 0.0 |
| L221Q | −3.05 | 42.4 | 5.2 |
| L221S | −3.46 | 23.6 | 0.0 |
| L221T | −3.93 | 9.7 | 0.0 |
| L221W | 1.08 | 21.5 | 0.0 |
| L221Y | −3.48 | 29.6 | 5.2 |
| A223E | 0.53 | 1.2 | 0.0 |
| A223N | 2.18 | 43.2 | 5.2 |
| A223Q | 2.30 | 17.2 | 5.2 |
| Q224D | −1.34 | 9.4 | 5.2 |
| Q224E | 1.70 | 24.8 | 5.2 |
| Q224G | −0.73 | 29.5 | 5.2 |
| G226D | −1.04 | 17.4 | 0.0 |
| G226E | 0.22 | 20.9 | 0.0 |

B.iii. Less immunogenic variants of CTRP1 agretope B16.

| | | | |
|---|---|---|---|
| wt | 0.00 | 34.5 | 0.0 |
| I230A | −23.31 | 0.0 | 0.0 |
| I230D | −21.92 | 0.0 | 0.0 |
| I230E | −8.53 | 0.0 | 0.0 |
| I230G | −20.49 | 0.0 | 0.0 |
| I230H | −9.15 | 0.0 | 0.0 |
| I230K | 1.87 | 0.0 | 0.0 |
| I230Q | −4.26 | 0.0 | 0.0 |
| M231A | −1.21 | 30.3 | 0.0 |
| M231D | −0.95 | 6.1 | 0.0 |
| M231E | −1.88 | 30.3 | 0.0 |
| M231G | −0.12 | 30.3 | 0.0 |
| M231N | −5.76 | 30.3 | 0.0 |
| M231S | −4.58 | 15.2 | 0.0 |
| M231T | −6.32 | 30.3 | 0.0 |
| Q232D | 4.45 | 15.2 | 0.0 |
| Q232E | 0.81 | 30.3 | 0.0 |
| S233A | 0.32 | 20.1 | 0.0 |
| S233G | 2.18 | 10.2 | 0.0 |
| S233P | 0.87 | 10.2 | 0.0 |
| S235A | 0.98 | 30.3 | 0.0 |
| S235E | 0.55 | 0.0 | 0.0 |
| S235K | 1.42 | 15.2 | 0.0 |
| S235P | 0.01 | 30.3 | 0.0 |
| S235Q | 1.89 | 15.2 | 0.0 |
| S235T | 1.41 | 32.9 | 0.0 |
| L236A | −7.37 | 15.2 | 0.0 |
| L236D | −6.40 | 0.0 | 0.0 |
| L236E | −7.42 | 15.2 | 0.0 |
| L236G | −5.72 | 15.2 | 0.0 |
| L236H | 0.58 | 15.2 | 0.0 |
| L236I | −3.38 | 30.3 | 0.0 |
| L236M | −0.99 | 30.3 | 0.0 |
| L236P | −7.09 | 6.1 | 0.0 |
| L236Q | −4.07 | 30.3 | 0.0 |
| L238A | −6.63 | 3.4 | 0.0 |
| L238D | −3.93 | 0.0 | 0.0 |
| L238E | −2.41 | 0.0 | 0.0 |
| L238G | −5.09 | 0.0 | 0.0 |
| L238K | −8.96 | 3.4 | 0.0 |
| L238M | −8.07 | 17.8 | 0.0 |
| L238N | −5.49 | 0.0 | 0.0 |
| L238Q | −6.02 | 10.2 | 0.0 |
| L238R | −3.44 | 0.0 | 0.0 |
| L238S | −6.00 | 11.1 | 0.0 |
| L238T | −5.86 | 0.0 | 0.0 |
| L238V | −3.47 | 15.2 | 0.0 |

B.iv. Less immunogenic variants of CTRP1 agretope B17.

| | | | |
|---|---|---|---|
| wt | 0.00 | 26.0 | 1.3 |
| W247A | −16.43 | 0.0 | 1.3 |
| W247D | −15.57 | 0.0 | 1.3 |
| W247E | −12.19 | 0.0 | 1.3 |
| W247G | −17.00 | 0.0 | 1.3 |
| W247K | −7.09 | 0.0 | 1.3 |
| W247L | −7.05 | 24.8 | 1.3 |
| W247M | −9.73 | 24.8 | 1.3 |
| W247N | −14.85 | 0.0 | 1.3 |
| W247P | −14.01 | 0.0 | 1.3 |
| W247Q | −10.17 | 0.0 | 1.3 |
| W247S | −16.77 | 0.0 | 1.3 |
| V248A | −3.93 | 0.0 | 0.0 |
| V248G | −1.67 | 2.8 | 0.0 |
| V248M | −6.88 | 7.0 | 1.3 |
| V248P | 0.21 | 0.0 | 0.0 |
| V248S | −3.71 | 0.0 | 0.0 |
| V248T | −2.74 | 0.0 | 0.0 |
| R249A | −0.61 | 8.9 | 0.0 |
| R249D | 1.48 | 0.0 | 0.0 |
| R249E | 1.02 | 0.0 | 0.0 |
| R249G | 1.61 | 12.9 | 0.0 |
| R249K | −2.89 | 8.9 | 0.0 |
| R249N | −0.33 | 24.5 | 0.0 |
| R249P | 0.46 | 12.9 | 0.0 |
| R249Q | −2.30 | 8.9 | 0.0 |
| R249S | −2.85 | 12.9 | 0.0 |
| R249T | −2.07 | 8.9 | 0.0 |
| R249V | −1.09 | 24.5 | 0.0 |
| L250A | −6.84 | 16.1 | 0.0 |
| L250E | −8.85 | 0.0 | 0.0 |
| L250G | −5.33 | 13.1 | 0.0 |
| L250K | −6.66 | 19.0 | 0.0 |
| L250N | −7.81 | 22.4 | 0.0 |
| L250P | −0.70 | 0.0 | 0.0 |
| L250Q | −8.64 | 12.2 | 0.0 |
| L250S | −7.10 | 13.1 | 0.0 |
| L250T | −8.72 | 2.8 | 0.0 |
| L250V | −7.77 | 12.5 | 0.0 |
| K252A | 1.51 | 13.7 | 1.3 |
| K252D | 1.69 | 0.0 | 1.3 |
| K252E | 0.87 | 0.0 | 1.3 |
| K252F | 3.71 | 0.0 | 1.3 |
| K252G | 1.72 | 7.6 | 1.3 |
| K252H | 1.02 | 2.8 | 1.3 |
| K252L | −0.53 | 7.0 | 1.3 |
| K252N | 0.48 | 4.5 | 1.3 |
| K252P | 0.55 | 15.4 | 1.3 |
| K252Q | 1.09 | 2.8 | 1.3 |
| K252R | 0.02 | 24.8 | 1.3 |
| K252S | 0.92 | 8.8 | 1.3 |
| K252T | 0.47 | 22.6 | 1.3 |
| K252W | 3.20 | 0.0 | 1.3 |
| K252Y | 3.32 | 0.0 | 1.3 |
| G253E | 3.66 | 21.5 | 0.0 |
| R255D | −0.55 | 12.9 | 1.3 |
| R255E | 0.18 | 14.7 | 1.3 |
| R255H | 1.29 | 21.9 | 1.3 |
| R255K | −3.73 | 20.8 | 1.3 |
| R255L | 1.68 | 24.7 | 1.3 |
| R255N | −1.10 | 19.5 | 1.3 |
| R255P | −0.99 | 19.4 | 1.3 |
| R255T | −0.96 | 20.5 | 1.3 |

While the foregoing invention has been described above, it will be clear to one skilled in the art that various changes and additional embodiments made be made without departing from the scope of the invention. All references cited herein, including patents, patent applications (provisional, utility and PCT), and publications are entirely incorporated by reference in their entirety.

The analyses were performed on the ordered, extracellular domains (underlined) of the C1q superfamily members, as determined via examination of Protein Data Bank structures.

Adiponectin (SEQ_ID:1)
MLLLGAVLLLLALPGHDQETTTQGPGVLLPLPKGACTGWMAGIPGHPGHN
GAPGRDGRDGTPGEKGEKGDPGLIGPKGDIGETGVPGAEGPRGFPGIQGR
KGEPGEGAYVYRSAFSVGLETYVTIPNMPIRFTKIFYNQQNHYDGSTGKF
HCNIPGLYYFAYHITVYMKDVKVSLFKKDKAMLFTYDQYQENNVDQASG
SVLLHLEVGDQVWLQVYGEGERNGLYADNDNDSTFTGFLLYHDTN
CTRPI (SEQ_ID:2):
MGSRGQGLLLAYCLLLAFASGLVLSRVPHVQGEQQEWEGTEELPSPPDHA
EPAEEQHEKYRPSQDQGLPASRCLRCCDPGTSMYPATAVPQINITILKGE
KGDRGDRGLQGKYGKTGSAGARGHTGPKGQKGSMGAPGERCKSHYAAFSV
GRKKPMHSNHYYQTVIFDTEFVNLYDHFNMFTGKFYCYVPGLYFFSLNVH
TWNQKETYLHIMKNEEEVVILFAQVGDRSIMQSQSLMLELREQDQVWVRL
YKGERENAIFSEELDTYITFSGYLVKHATEA

1. A non-naturally occurring variant adiponectin protein having reduced immunogenicity as compared with a wild type adiponectin protein (SEQ. ID. 1), wherein said variant protein comprises at least two amino acid modifications.

2. A variant protein of claim 1 wherein at least one amino acid modification is made to the group consisting of Agretope A1 (residues 109-117), Agretope A2 (residues 111-119), Agretope A3 (residues 122-130), Agretope A4 (residues 128-136), Agretope A5 (residues 136-144), Agretope A6 (residues 157-165), Agretope A7 (residues 158-166), Agretope A8 (residues 160-168), Agretope A9 (residues 166-174), Agretope A110 (residues 167-175), Agretope A111 (residues 175-183), Agretope A12 (residues 176-184), and Agretope A13 (residues 202-210).

3. A variant protein of claim 1, wherein at least one modification is selected from the group consisting of positions 109, 110, 111, 112, 113, 114, 115, 117, 119, 122, 123, 124, 125, 127, 128, 130, 166, 167, 168, 169, 171, 172, and 174; and wherein the possible modifications at position 109 are selected from the group consisting of Q, H, N, R, E, K, G, A, D, T, S, L, P, V, M, and I; wherein the possible modifications at position 110 are selected from the group consisting of Q, K, N, L, D, A, E, T, S, P, Y, H, F, G, M, I, and W; wherein the possible modifications at position 111 are selected from the group consisting of P, N, H, A, W, S, D, R, K, T, E, Q, G, and V; wherein the possible modifications at position 112 are selected from the group consisting of K, D, and G; wherein the possible modification at position 113 is A; wherein the possible modification at position 114 is G; wherein the possible modifications at position 115 are selected from the group consisting of Y, H, and M; wherein the possible modifications at position 117 are selected from the group consisting of T, S, and A; wherein the possible modifications at position 119 are selected from the group consisting of Q, K, E, T, S, R, A, N, and D; wherein the possible modifications at position 122 are selected from the group consisting of K, E, H, D, R, and Q; wherein the possible modifications at position 123 are selected from the group consisting of 1, P, E, T, A, Q, S, and N; wherein the possible modifications at position 124 are selected from the group consisting of E and D; wherein the possible modifications at position 125 are selected from the group consisting of E, D, Y, Q, N, S, V, T, L, H, K, R, W, F, G, and P; wherein the possible modifications at position 127 are selected from the group consisting of K, D, and G; wherein the possible modifications at position 128 are selected from the group consisting of V, T, Q, A, S, H, N, L, E, D, I, G, K, and R; wherein the possible modifications at position 130 are selected from the group consisting of L, V, N, Q, E, M, D, and A; wherein the possible modifications at position 166 are selected from the group consisting of T, S, and A; wherein the possible modifications at position 167 are selected from the group consisting of F, H, A, E, G, D, and W; wherein the possible modifications at position 168 are selected from the group consisting of A and G; wherein the possible modifications at position 169 are selected from the group consisting of A, E, W, and T; wherein the possible modifications at position 171 are selected from the group consisting of T, A, S, G, and N; wherein the possible modifications at position 172 are selected from the group consisting of R, Q, G, E, and D; wherein the possible modifications at position 174 are selected from the group consisting of A and G.

4. A variant protein of claim 1, wherein at least one modification is selected from the group consisting of positions 109, 110, 111, 112, 122, and 166; and wherein the possible modifications at position 109 are selected from the group consisting of Q, H, N, R, E, K, G, A, D, T, S, and P; wherein the possible modifications at position 110 are selected from the group consisting of D, A, E, T, S, P, G, and W; wherein the possible modifications at position 111 are selected from the group consisting of P, N, H, A, S, D, R, K, T, E, Q, and G; wherein the possible modification at position 112 is D; wherein the possible modification at position 122 are selected from the group consisting of K, E, H, D, R, Q; and wherein the possible modifications at position 166 are selected from the group consisting of T, S, and A.

5. A non-naturally occurring variant CTRP1 protein having reduced immunogenicity as compared with a wild type CRTP-1 protein (SEQ. ID. 2), wherein said variant protein comprises at least two amino acid modifications.

6. A variant protein of claim 5, wherein at least one amino acid modification is made to the group consisting of Agretope B1 (residues 150-158), Agretope B2 (residues 171-179), Agretope B3 (residues 172-180), Agretope B4 (residues 178-186), Agretope B5 (residues 185-193), Agretope B6 (residues 186-194), Agretope B7 (residues 188-196), Agretope B8 (residues 192-200), Agretope B9 (residues 193-201), Agretope B10 (residues 194-202), Agretope B11 (residues 202-210), Agretope B12 (residues 208-216), Agretope B13 (residues 209-217), Agretope B14 (residues 218-226), Agretope B15 (residues 220-228), Agretope B16 (residues 230-238), Agretope B17 (residues 247-255), Agretope B18 (residues 248-256), Agretope B19 (residues 267-275).

7. A variant protein of claim 5, wherein at least one modification is selected from the group consisting of positions 172, 174, 175, 177, 178, 180, 218, 219, 220, 221, 223, 224, 226, 230, 231, 232, 233, 235, 236, 238, 247, 248, 249, 250, 252, 253, and 255; and wherein the possible modifications at position 172 are selected from the group consisting of A, D, G, N, S, and T; wherein the possible modifications at position 174 are selected from the group consisting of A, D, E, G, H, K, N, P, Q, R, S, T, V, and W; wherein the possible modifications at position 175 are selected from the group consisting of A, D, E, G, I, K, L, M, N, P, and T; wherein the possible modifications at position 177 are selected from the group consisting of D, E, G, and Q; wherein the possible modifications at position 178 are selected from the group consisting of H, K, and Y; wherein the possible modifications at position 180 are selected from the group consisting of A, D, E, F, G, H, K, L, N, P, Q, R, S, T, V, W, and Y; wherein the possible modifications at position 218 are selected from the group consisting of A, G, N, S, and T; wherein the possible modifications at position 219 are selected from the group consisting of A, D, E, G, H, I, K, L, M, N, Q, S, and T; wherein the possible modifications at position 220 are selected from the group consisting of A, D, G, L, N, S, T, and V; wherein the possible modifications at position 221 are selected from the group consisting of A, D, E, F, G, I, K, N, P, Q, S, T, W, and Y; wherein the possible modifications at position 223 are selected from the group consisting of E, N, and Q; wherein the possible modifications at position 224 are selected from the group consisting of D, E, and G; wherein the possible modifications at position 226 are selected from the group consisting of D and E; wherein the possible modifications at position 230 are selected from the group consisting of A, D, G, H, E, Q, and K; wherein the possible modifications at position 231 are selected from the group consisting of T, N, S, E, A, D, and G; wherein the possible modifications at position 232 are selected from the group consisting of E and D; wherein the possible modifications at position 233 are selected from the group consisting of A, P, and G; wherein the possible modifications at position 235 are selected from the group consisting of P, E, A, T, K, and Q; wherein the possible modifications at position 236 are selected from the group consisting of E, A, P, D, G, Q, I, M, and H; wherein the possible modifications at position 238 are selected from the group consisting of K, M, A, Q, S, T, N, G, D, V, R, and E; wherein the possible modifications at position 247 are selected from the group consisting of G, S, A, D, N, P, E, Q, M, K, and L; wherein the possible modifications at position 248 are selected from the group consisting of M, A, S, T, G, and P; wherein the possible modifications at position 249 are selected from the group consisting of K, S, Q, T, V, A, N, P, E, D, and G; wherein the possible modifications at position 250 are selected from the group consisting of E, T, Q, N, V, S, A, K, G, and P; wherein the possible modifications at position 252 are selected from the group consisting of L, R, T, N, P, E, S, H, Q, A, D, G, W, Y, and F; wherein the possible modification at position 253 is E; wherein the possible modifications at position 255 are selected from the group consisting of K, N, P, T, D, E, H, and L.

8. A variant protein of claim 5, wherein at least one modification is selected from the group consisting of positions 172, 174, 175, 218, 219, 220, 230, 235, 236, 238, 247, 248, 249, 250 and 252; and wherein the possible modifications at position 172 are selected from the group consisting of A, D, G, N, S, and T; wherein the possible modifications at position 174 are selected from the group consisting of D and E; wherein the possible modifications at position 175 are selected from the group consisting of E and P; wherein the possible modifications at position 218 are selected from the group consisting of A, G, N, S, and T; wherein the possible modification at position 219 is D; wherein the possible modification at position 220 is D; wherein the possible modifications at position 230 are selected from the group consisting of A, D, G, H, E, Q, and K; wherein the possible modification at position 235 is E; wherein the possible modification at position 236 is D; wherein the possible modifications at position 238 are selected from the group consisting of T, N, G, D, R, and E; wherein the possible modifications at position 247 are selected from the group consisting of G, S, A, D, N, P, E, Q, and K; wherein the possible modifications at position 248 are selected from the group consisting of A, S, T, and P; wherein the possible modifications at position 249 are selected from the group consisting of E and D; wherein the possible modifications at position 250 are selected from the group consisting of E and P; wherein the possible modifications at position 252 are selected from the group consisting of E, D, W, Y, and F.
